(19) United States
(12) Patent Application Publication
(10) Pub. No.: US 2017/0204445 A1
(43) Pub. Date: Jul. 20, 2017

(54) IN VIVO SITE-SPECIFIC PROTEIN TAGGING USING ENGINEERED SORTASE VARIANTS

(71) Applicant: The Board of Trustees of the Leland Stanford Junior University, Stanford, CA (US)

(72) Inventors: Jeff E. Glasgow, Millbrae, CA (US); Marc L. Salit, San Carlos, CA (US); Jennifer R. Cochran, Stanford, CA (US)

(21) Appl. No.: 15/406,247

(22) Filed: Jan. 13, 2017

Related U.S. Application Data
(60) Provisional application No. 62/279,608, filed on Jan. 15, 2016.
(60) Provisional application No. 62/345,233, filed on Jun. 3, 2016.

(51) Int. Cl. C12P 21/00; C07K 16/32; C12N 9/52

(52) U.S. Cl. CPC C12P 21/00; C12N 9/52; C12Y 304/2207; C07K 16/32; C07K 2317/40; C07K 2317/515; C07K 2317/51

(57) ABSTRACT

Engineered sortase variants are shown to have promiscuous activity that allow proteins to be tagged using a diverse array of small, commercially available amines, including bioorthogonal functional groups. This technique can also be carried out in living microbial cells, enabling simple, inexpensive production of chemically functionalized proteins with no additional purification steps. The methods find use in the site specific conjugation of drugs, imaging probes and other chemical moieties to proteins and peptides.

### CROSS REFERENCE

This application claims benefit of U.S. Provisional Patent Application No. 62/279,608 filed Jan. 15, 2016 and U.S. Provisional Patent Application No. 62/345,233, filed Jun. 3, 2016, which applications are incorporated herein by reference in their entirety.

### BACKGROUND

Site-specific modification of proteins is a longstanding challenge in the pharmaceutical and biotechnology arts. The classic methods oftentimes lead to non-specific labeling (e.g. NHS Lys labeling) or require engineering (e.g. maleimide Cys labeling or unnatural amino acids). In addition, the repertoire of selective chemical reactions, however, is very limited. One alternative is, by recombinant methods, to introduce special unnatural amino acids having a unique reactivity and then exploit this reactivity in the further derivatization. Another alternative is the use of enzymes which recognize structural and functional features of the protein to be modified.

The present invention relates generally to a novel method of introducing modifying groups to a protein. Chemoenzymatic modification of proteins is an attractive option to create highly specific conjugates in gentle, biological conditions. However, these methods often suffer from expensive specialized substrates, bulky fusion tags, low yields, and extra purification steps to achieve the desired conjugate. Staphylococcus aureus sortase A and its engineered variants have been used to attach oligoglycine derivatives to the C-terminus of proteins expressed with a minimal LPXTG tag. This strategy has been used extensively for bioconjugation in vitro, and for protein-protein conjugation in living cells.

The selective derivatization of proteins remains a very difficult task. Accordingly, there is a need in the art for methods of selectively derivatizing amino acid residues in proteins or polypeptides.

### SUMMARY

Methods and compositions for modifying a protein are provided. The method permits site selective modifications. Engineered sortase variants are shown to have promiscuous activity that allow proteins to be tagged using a diverse array of small, commercially available amines, including several bioorthogonal functional groups. This technique can also be carried out in living microbial cells, enabling simple, inexpensive production of chemically functionalized proteins with no additional purification steps. The methods find use in the site specific conjugation of drugs, imaging probes and other chemical moieties to proteins and peptides.

Methods of the invention are useful in conjugating bioorthogonal functional groups in gentle, protein-friendly conditions. When coexpressed with a target LPETG-tagged proteins, sortase is able to conjugate both azide and alkyne functional groups to the target proteins as they are expressed in living cells. The azide group can then be labeled in cell lysate before protein purification, resulting in a robust, simple protocol for producing site-specific protein conjugates. The target protein may be, without limitation, growth factors, cytokines, chemokines, antigens, e.g. for vaccine production, biologically active peptides, antibodies, antibody fragments, and the like.

Methods are also provided for living cell chemical biology experiments requiring in vivo labeling with organic fluorophores or affinity handles.

In some embodiments, the methods of the invention comprise tagging a target protein at the carboxy terminus with an LPXTG sortase tag. The sortase tag may be LPETG. The sortase tagged target protein is contacted with an engineered sortase enzyme, including without limitation SrtA7M or a derivative thereof, as defined herein. The contacting is performed in the presence of a substrate, which substrates comprise an amine moiety for conjugation. Non-limiting examples of amines suitable for this purpose are shown below (compounds 1-9), and analogs and derivatives thereof. In some embodiments, the amine substrate provides a reactant group for click chemistry modification of the protein.

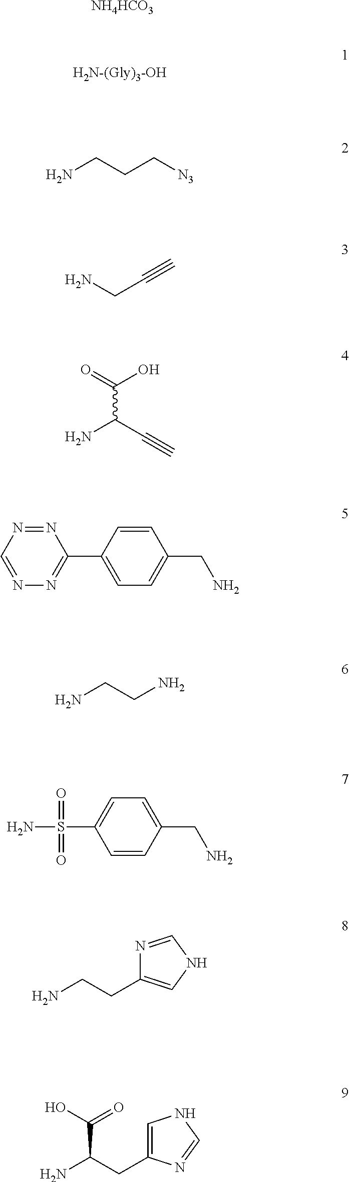

In some embodiments, the contacting step is performed with a cell lysate. In some embodiments, the reaction is buffered with tris, in other embodiments the reaction if buffered with ammonium bicarbonate. In some embodiments the pH of the reaction is greater than about 7.5. In some embodiments the pH of the reaction is from about pH 7.5 to about pH 9, including from about 7.5 to about 8.5, from about 7.5 to about 8. In some embodiments, the contacting is performed in a living cell, e.g. a bacterial cell.

In some embodiments, a system is provided for yeast surface display where yeast-displayed protein variants are fused to two different proteins to Aga2p, one to the N-terminus and one to the C-terminus. Using this approach allows an antibody fragment, ligand, or receptor to be directly coupled to expression of a fluorescent protein readout, eliminating the need for antibody-staining of epitope tags to quantify yeast protein expression levels. This system simplifies quantification of protein-protein binding interactions measured on the yeast cell surface.

In some embodiments, a bioconjugation enzyme and its corresponding peptide substrate are co-expressed on the same Aga2p construct, enabling enzyme expression and catalytic activity to be measured on the surface of yeast. In some embodiments the bioconjugation enzyme is a sortase enzyme. In some embodiments a bioorthogonal functional group is conjugated to a peptide substrate on the yeast-displayed protein variant. Such a dual protein display system can provide for measurement of enzyme-mediated bioconjugation on the surface of yeast.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. C terminal transpeptidation of proteins using sortase A. (A) Classic sortase reaction. The enzyme recognizes a LPXTG motif on the protein of interest (POI) and replaces the terminal glycine with tagged oligoglycine 1. (B) New sortase activity where oligoglycine is replaced by an inexpensive amine containing a cell-permeable, bioorthogonal chemical handle.

FIG. 2. Transpeptidation of proteins using SrtA7M. Peptide LPETGSW (1 mM, dashed line) was incubated with SrtA7M (20 μM) and amines 1-9 (10 mM) at 37° C. for 2 hours in ammonium bicarbonate buffer pH 7.8, diluted 10-fold with 0.2% formic acid and analyzed by LC-quadrupole MS. Negative mode, base-peak chromatograms show significant conversion to the desired conjugates, indicated by (*). Ammoniolysis occurs in the absence of unbranched primary amines.

FIG. 3. Transpeptidation of purified proteins. Purified proteins expressed with a C-terminal LPETGG sequence were incubated with 10 μM SrtA7M for 8 hours at 37° C. along with the desired amine. Proteins were then diluted 10-fold with 0.2% formic acid and analyzed by ESI-TOF LC-MS. (A) Maltose binding protein. (B) Sf9 nanobody, and (C) Fibronectin Fn10, all show 80-100% conversion to the desired conjugate.

FIG. 4. Modification of proteins in living E. coli cells. SrtA7M and each protein of interest were co-expressed and amine (25 mM) was added to the culture medium. Clarified lysate was then incubated with Cy3-DBCO. (A) Cy3-fluorescence and (B) Coomassie-stained SDS_PAGE gels show high levels of modification of sfGFP, GST. Trx-5s7 nanobody (before and after Ni++/NTA purification and MBP. (C) Mass spectra of superfolder GFP expressed alone or in combination with SrtA7M and Azp or propargylamine and purified by Ni++/NTA chromatography, showing complete conversion to the desired conjugates. (D) Flow cytometry with Trx-5f7-Cy3. Trx-5f7 was conjugated to Azp in vivo, labeled with Cy3-DBCO on the Ni++/NTA column, and bound to HER2 molecules on SK-BR-3 cells. Blocked cells were incubated with 100-fold excess unlabeled Trx-5f7 to discern non-specific Cy3 binding to the cell

FIG. 5. Buffer and amine screen. Peptide LPETGSW (1 mM) was incubated with SrtA7M (20 μM) for 4 hours at 37° C. with amines in either 100 mM sodium phosphate buffer pH 7.4 or Tris.HCl pH 8.0, followed by quenching with 9 volumes of 0.8% formic acid and analysis by negative-mode, quadrupole LC-MS. The enzyme shows poor conversion in phosphate buffer with Gly3, and little to no conversion with other amines (not shown). Significant conversion is seen in Tris buffer, but less than in ammonium bicarbonate, particularly with amines 2 and 5.

FIG. 6. Sortase 7M traspeptidation with propargylamine is sensitive to pH. Enzyme (10 μM) was incubated with 1 mM LPETGSW and 10 mM propargylamine in 100 mM Tris.HCl at the pH indicated for 2 hours at 37° C. followed by LC/MS analysis.

FIG. 7. Transpeptidation of peptides with wild-type Staphylococcus aureus sortase (A) A LC/MS analysis of WT SrtA transpeptidation of LPETGSW peptide in vitro. SrtA (10 or 150 μM) was incubated with peptide (1 mM) and amine (10 mM) in 25 mM Tris.HCl pH 7.5, 150 mM NaCl, and 10 mM CaCl2 for 2 or 20 hours at 37° C. The reaction was quenched with 9 volumes of 0.8% formic acid and analyzed by LC/MS. Negative-mode base-peak chromatograms indicate significant labeling of peptide as denoted by asterisks (*). (B) Mass spectrum of Gly₃-modified peptide. C. Mass spectrum of propargylamine-modified peptide.

FIG. 8. Azp concentration screen. MBP (100 μM was incubated with SrtA7M (10 μM) for 8 hours at 37° C. with varying concentrations of Azp, followed by quenching with 9 volumes of 0.8% formic acid. Lower concentrations show almost complete ammoniolysis, while higher concentrations show high levels of Azp conjugation.

FIG. 9. Sortase conjugation time course. MBP (100 μM was incubated with SrtA7M (100 μM) at 37° C. with Azp (100 mM). Aliquots were taken at each time point and quenched with 9 volumes of 0.8% formic acid. The reaction is nearly complete after only 1 hour, with only slight increases in yield in subsequent time points.

FIG. 10. In vitro conjugation of sfGFP derivatives. (A) His-sfGFP-srt and (B) sfGFPHis-srt were incubated with 10 μM SrtA7M and 100 mM Azp or propargylamine for 8 hours at 37° C. No detectable modification of His-sfGFP-srt took place, but sfGFP-His-srt was completely modified with the desired amines.

FIG. 11. Specificity of labeling in vivo. GST-His-srt, GFPHis-srt, and MBP-srt were coexpressed with SrtA7M in the presence of Azp. After 22 hours cells were washed and lysed. Lysate was incubated with biotin DIBAC, run on a 12% SDS-PAGE gel, transferred to a nitrocellulose membrane, and stained with Neutravidin-horseradish peroxidase. Lanes: 1,4: GST-His-srt 2, 5: GFP-His-srt and 3, 6: MBP-srt.

FIG. 12. Sortase expression screen. Cells containing plasmids for sfGFP-His-srt and sortase were induced with varying concentrations of rhamnose, along with S mM Azp. After growth and labeling overnight at 30° C., cells were lysed and clarified, then labeled with 500 μM biotin-DIBAC for 3 hours. (A) Western blot of biotin-DIBAC-labeled protein in lysate, detected using Neutravidin-HRP. Saturating induction of sortase did not increase labeling efficiency over subsaturating rhamnose. (B) Gel image was processed in Image] to quantify band intensity.

FIG. 13. In vivo conjugation of sfGFP derivatives. (A) Amine (25 mM) added to culture diffuses into cells coexpressing sortase and the protein of interest. (B) sfGFP-His-srt was expressed without sortase or added amine. (C) sfGFP-His-srt was coexpressed with SrtA7M without added amine. Several unidentified adducts are visible on the mass spectrum. (D) sfGFP-His-srt was coexpressed with SrtA7M with Gly3 added to the culture. (E) sfGFP-His-srt was coexpressed with SrtA7M with propargylamine added to the culture. (F) sfGFP-His-srt was co expressed with SrtA7M with Azp added to the culture. B, E, and F appear in FIG. 4C, and are expanded here for detail.

FIG. 14. Sortase-based modification is useful for attaching cargo to antibodies in high yield with site-specificity.

FIG. 15. Schematic of yeast surface display strategies. (A) Conventional vectors, pTMY (N-terminal Aga2p fusion) and pCT (C-terminal Aga2p fusion). (B) pCL, a vector that enables co-expression of two proteins on the N- and C-terminus of the Aga2p subunit. (C) The expression cassette of pCL in which one protein is inserted between the synthetic prepro signal peptide and Aga2p and the other protein is inserted downstream of Aga2p. Various epitope tags (HA, c-Myc and FLAG) are included to validate and compare protein expression across formats.

FIG. 16. Quantification of protein-protein interactions on yeast using pCL vectors. (A) Schematic of protein display and antibody-staining strategies for the pCL vectors co-expressing a protein-of-interest and yEGFP on the same surface. (B) Binding curves comparing pCL and pCT/pTMY vectors for a lysozyme-binding scFv antibody fragment (left), a Gas6-binding Axl Ig1 receptor domain (middle), and the Met-binding NK1 ligand (right). Error bars correspond to the standard deviation of three independent measurements. N.D.: Binding was not able to be determined for pTMY-NK1 due to poor yeast expression. (C) Equilibrium binding constants, KD, of yeast-displayed proteins expressed with the pCL or pCT/pTMY vectors. (D) Wild-type proteins (Axl and NK1) and engineered variants (MYD1 [36] and M2.2 [24]), expressed using pCL vectors, can be differentiated at low target concentrations (0.1 nM Gas6 and 0.5 nM Met-Fc) on flow cytometry scatter plots.

FIG. 17. Evaluation of bioconjugation enzyme activity on yeast using pCL vectors. (A, B) Schematic of protein expression and bioconjugation reactions on the yeast surface for pCL vectors co-expressing sortase A (SrtA) 7M, and its peptide substrate, LPETGG. (C) Detection of SrtA bioconjugation activity on yeast using pCL-Srt-LS (long linker) and pCL-Srt-SS (short linker) under various conditions. Samples were stained with avidin-PE to detect Azp conjugation followed by biotin Click chemistry, and also stained with AlexaFluor 488-conjugated antibodies to measure yeast expression levels. Histograms of PE signal (enzyme activity) as a percentage of the maximum cell number shows readily apparent sortase-active populations in each sample. (D) Detection of SrtA bioconjugation activity on yeast by using pCL-Srt-cGFP-LS (yEGFP plus long linker) with and without biotin-DBCO. Co-expression of yEGFP and SrtA eliminates the need for antibody-staining of epitope tags to quantify yeast surface expression levels.

FIG. 18. Yeast-codon-optimized enhanced GFP (yEGFP) expression when fused at the C-terminus (A) and N-terminus (B) of Aga2p under different induction temperatures (20° C. or 30° C.). Yeast cells were stained with anti-c-Myc primary antibody (Thermo Fisher Scientific, A21281) followed by PE-labeled secondary antibody (Santa Cruz, sc-3730) to measure c-Myc expression levels.

FIG. 19. Schematic diagrams and plasmid maps of pCL-nGFP (A) and pCL-cGFP (B). The Axl Ig1 domain (Axl) was cloned into pCL-nGFP and the NK1 domain of human HGF was cloned into pCL-cGFP as model proteins for binding assays.

FIG. 20. Comparison of scFv D1.3 expression using pCT (A), pCL-cGFP (B), and pCL-nGFP (C) at different induction temperatures. C-terminal expression of the scFv D1.3 protein and 20° C. induction conditions were deemed most optimal.

FIG. 21. (A) Antibody staining strategy for proteins expressed using pCL-nGFPAga2p-Axl (pCL-Axl). (B) Flow cytometry scatter plots of negative controls and cells incubated with different Gas6 concentrations ranging from 50 pM to 100 nM. In each scatter plot, the GFP-positive population is gated to calculate a geometric mean of binding signal at the designated Gas6 concentration.

FIG. 22. (A) A distinct NK1-expressing yeast population is observed through GFP-expression of the pCL vector compared to low NK1 expression observed with the pTMY vector, measured through the HA epitope tag. Expression signals were measured after incubating the yeast cells to bind 500 nM Met-Fc. (B) For pTMY-NK1, the binding affinity of NK1 against Met-Fc was not measurable in the titration curve. Two independent experiments are plotted.

FIG. 23. Yeast-displayed yEGFP expression correlates with epitope tag expression on the same yeast cell and its signal intensity remains constant over time. (A) yEGFP fluorescence measured in yeast cells transformed with pCL-nGFP-Aga2p-Axl (pCL-Axl), correlated with fluorescence of C-terminal c-Myc expression measured by antibody staining. Cells were incubated with PBSA containing chicken anti-c-Myc antibody for 30 min at 4° C., washed with 1 ml of PBSA, and then stained with PBSA containing AlexaFluor 555 goat anti-chicken IgY for 20 min at 4° C. R² was calculated from simple linear regression of all the clones in an expression positive population. (B) Comparison of expression signals between Axl Ig1 displayed using pCT or pCL vectors upon increasing the number of antibody-staining/washing steps. In each group, signals are normalized to the fluorescence intensity of the original condition. Error bars correspond to the standard deviation of six independent measurements (n=6 for each of pCT and pCL groups). One-tailed, paired Student's t-tests were performed to analyze significance of the expressing signal change. **p≦0.01; n.s. for p>0.05. (C) yEGFP yeast surface expression levels measured over 70 h incubation of yeast at room temperature. In these studies, the Axl Ig1 protein is expressed using the pCT or the pCL-nGFP-Aga2p-Axl vector. Error bars correspond to the standard deviation of triplicate samples.

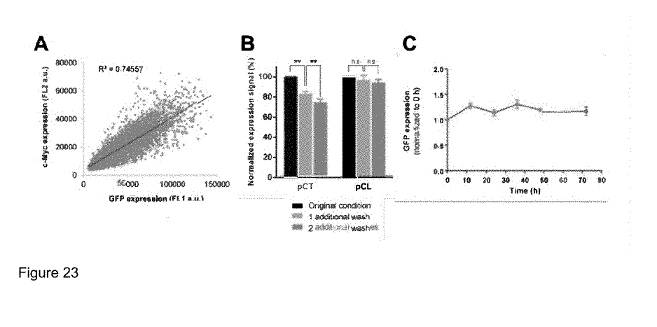

FIG. 24. Flow cytometry scatter plots under various conditions for sortase-LPETGG bioconjugation using pCL-Srt-LS (substrate tethered using a long linker), pCL-Srt-SS (substrate tethered using a short linker), and pCL-Srt-cGFP-LS (substrate tethered through C-terminal GFP plus a long linker).

FIG. 25. (A) (B) Comparison between the original pCL vector (SEQ.1) and pCL2 (SEQ.2), which have been optimized for facile and modular cloning. Modified features are highlighted in red blocks (codon-optimized linkers) and ovals (restriction site additions and modifications).

### DETAILED DESCRIPTION

All references made to patents, patent publications, and other literature are made for their incorporation into this disclosure to the extent permissible by law.

The present invention addresses the aforementioned needs by providing a method of introducing modifying compounds to a target protein in a selective manner via reaction with a modifying compound, while using conventional chemical methods. The resulting product is a protein having one or more groups capable of further chemical functionalization.

In one aspect, a process for modifying a protein includes: (a) forming an activated complex between an auxiliary protein and a modifying compound by catalytic action of microbial transglutaminase; (b) transferring the modifying compound from the activated complex to a target protein thereby creating a modified protein. As such, a “modified protein” as used herein, refers to a protein or polypeptide that has been selectively modified by addition of a modifying compound using microbial transglutaminase.

Sortase refers to a group of prokaryotic enzymes that modify surface proteins by recognizing and cleaving a carboxyl-terminal sorting signal. For most substrates of sortase enzymes, the recognition signal consists of the motif LPXTG (Leu-Pro-any-Thr-Gly), then a highly hydrophobic transmembrane sequence, followed by a cluster of basic residues such as arginine. Cleavage occurs between the Thr and Gly, with transient attachment through the Thr residue to the active site Cys residue, followed by transpeptidation that attaches the protein covalently to cell wall components. Sortases occur in almost all Gram-positive bacteria and the occasional Gram-negative (e.g. Shewanella putrefaciens) or Archaea (e.g. Methanobacterium thermoautotrophicum).

This group of cysteine peptidases belong to MEROPS peptidase family C60 (clan C-) and include the members of several subfamilies of sortases. Another sub-family of sortases (C60B in MEROPS) contains bacterial sortase B proteins that are approximately 200 residues long.

The transpeptidase activity of sortase is taken advantage to produce conjugate polypeptides in vitro or in vivo. The recognition motif (LPXTG) is added to the C-terminus of a protein of interest. Upon addition of sortase to the protein, a substrate according to the invention is added to the N-terminus of the protein.

In certain embodiments the sortase enzyme has amino acid changes relative to the wild-type protein, [P94R/D160N/D165A/K190E/K196T] and [E105K/E108A], which sortase may be referred to SrtA7M. In some embodiments the sortase has the amino acid sequence:

MQAKPQIPKDKSKVAGYIEIPDADIKEPVYPGPATREQLNRGVSFAKENQ
SLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVKLE
HHHHHH

As used herein, the term “polypeptide” refers to a polymer of amino acid residues joined by peptide bonds, whether produced naturally or synthetically. Polypeptides of less than about 10 amino acid residues are commonly referred to as “peptides.” The term “peptide” is intended to indicate a sequence of two or more amino acids joined by peptide bonds, wherein said amino acids may be natural or unnatural. The term encompasses the terms polypeptides and proteins, which may consist of two or more peptides held together by covalent interactions, such as for instance cysteine bridges, or non-covalent interactions.

A “protein” is a macromolecule comprising one or more polypeptide chains. A protein may also comprise non-peptidic components, such as carbohydrate groups. Carbohydrates and other nonpeptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless. A protein or polypeptide encoded by a non-host DNA molecule is a “heterologous” protein or polypeptide.

An “isolated polypeptide” is a polypeptide that is essentially free from cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, such as 96%, 97%, or 98% or more pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A target polypeptide is a substrate for sortase, by addition of a sortase tag. Addition of a tag can be brought about by standard techniques known to persons skilled in the art, such as genetic modification of the coding sequence. Target proteins can include enzymes, protein hormones, growth factors, antibodies and antibody fragments, cytokines, receptors, lymphokines and vaccine antigens. In some embodiments, the polypeptide is an antigenic peptide. In some embodiments the polypeptide is a carrier molecule, e.g. to enhance immunogenicity of a vaccine.

A polypeptide can be modified to alter the physico-chemical properties of the protein, such as e.g. to increase (or to decrease) solubility to modify the bioavailability of therapeutic proteins. In another embodiment, it may be desirable to modify the clearance rate in the body by conjugating compounds to the protein which binds to plasma proteins, such as e.g. albumin, or which increase the size of the protein to prevent or delay discharge through the kidneys. Conjugation may also alter and in particular decrease the susceptibility of a protein to hydrolysis, such as e.g. in vivo proteolysis.

In another embodiment, it may be desirable to conjugate a label to facilitate analysis of the protein. Examples of such labels include radioactive isotopes, fluorescent markers such as the fluorophores already described and enzyme substrates.

In still another embodiment, a compound is conjugated to a protein to facilitate isolation of the protein. For example, a compound with a specific affinity to a particular column material may be conjugated to the protein. It may also be desirable to modify the immunogenicity of a protein, e.g. by conjugating a protein so as to hide, mask or eclipse one or more immunogenic epitopes at the protein. The term “conjugate” as a noun is intended to indicate a modified peptide, i.e. a peptide with a moiety bonded to it to modify the properties of said peptide. As a verb, the term is intended to indicate the process of bonding a moiety to a peptide to modify the properties of said peptide.

In one embodiment, the invention provides a method of improving pharmacological properties of target proteins. The improvement is with respect to the corresponding unmodified protein. Examples of such pharmacological properties include functional in vivo half-life, immunogenicity, renal filtration, protease protection and albumin binding of any specific protein.

Bioconjugate chemistry has long been used to combine biomolecules to alter functions and to add properties. A variety of coupling methods have been developed to enable faster reaction kinetics, site-specificity, and robustness to changing environmental conditions. Of these methods, one of the most extensively utilized chemistries is “Click Chemistry” most notably, Huisgen copper(I)-catalyzed azide-alkyne cycloaddition. This reaction has a high thermodynamic driving force and is bioorthogonal. The method is enhanced for protein engineering purposes by the development of methods for reactions of azide and alkyne functional moieties.

Examples of amines that can be used in the methods of the invention include: tyrosine; glutamine; phenylalanine amino acid; serine amino acid; threonine amino acid; pyrrolysine amine; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amine, or any combination thereof or analog thereof; an amine with a photoactivatable cross-linker; a spin-labeled amine; a fluorescent amine; an amine with a novel functional group; an amine that covalently or noncovalently interacts with another molecule; a metal binding amine; a metal-containing amine; a radioactive amine; a photocaged and/or photoisomerizable amine; a biotin or biotin-analogue containing amine; a glycosylated or carbohydrate modified amine; a keto containing amine; amines comprising polyethylene glycol or polyether; a heavy atom substituted amine; a chemically cleavable or photocleavable amine; an amine with an elongated side chain; an amine containing a toxic group; a sugar substituted amine, e.g., a sugar substituted serine or the like; a carbon-linked sugar-containing amine; a redox-active amine; an α-hydroxy containing amine; an amine thio acid; an α,α disubstituted amine; a cyclic amine, etc.

Substrates may be selected to provide a reactant group for CLICK chemistry reactions (see Click Chemistry: Diverse Chemical Function from a Few Good Reactions Hartmuth C. Kolb, M. G. Finn, K. Barry Sharpless Angewandte Chemie International Edition Volume 40, 2001, P. 2004, herein specifically incorporated by reference), or for other bioorthogonal reactions. Other bioorthogonal chemistries such as the copper-free variant of this reaction (which uses a strained alkyne moiety), oxime formation between an acetyl group and an amino-oxy moiety, and a modified Staudinger ligation between an azide and a phosphine are also of interest.

### Pharmaceutical Compositions

In another aspect, pharmaceutical compositions comprising a protein modified by any of the methods disclosed herein The composition may further comprise a buffer system, preservative(s), tonicity agent(s), chelating agent(s), stabilizers and surfactants. In one embodiment, the pharmaceutical composition is an aqueous composition. Such compositions typically exist as a solution or a suspension. In a further embodiment, the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical composition is a freeze-dried composition, to which a physician, patient, or pharmacist adds solvents and/or diluents prior to use. In another embodiment the pharmaceutical composition is a dried composition (e.g. freeze-dried or spray-dried) ready for use without any prior dissolution.

In a further aspect, a pharmaceutical composition comprising an aqueous solution of a modified protein, e.g. where the protein is present in a concentration from 0.1-100 mg/ml or above, and wherein said composition has a pH from about 2.0 to about 10.0.

In a further embodiment, the buffer is selected from ammonium bicarbonate, sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)aminomethane, bicine, tricine, malic acid, succinate, maleic acid, fumaric acid, tartaric acid, aspartic acid, TRIS, or mixtures thereof.

In a further embodiment, the composition may also include a pharmaceutically acceptable preservative. For example, the preservative may be phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol), or mixtures thereof. The preservative may be present in a concentration from 0.1 mg/ml to 20 mg/m or 0.1 mg/ml to 5 mg/ml. In a further embodiment, the preservative is present in a concentration from 5 mg/ml to 10 mg/ml or from 10 mg/ml to 20 mg/ml.

In a further embodiment, the composition may include an isotonic agent. In a further embodiment, the isotonic agent is selected from a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. The use of an isotonic agent in pharmaceutical compositions is well-known to the skilled person. For convenience, reference is made to Remington: The Science and Practice of Pharmacy, 201h edition, 2000.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenes-ulfonic acids and the like. Further examples of pharmaceutically acceptable inorganic or organic acid addition salts include the pharmaceutically acceptable salts listed in J. Phann. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In a further embodiment, the composition includes a chelating agent. The chelating agent is selected from salts of ethylenediaminetetraacetic acid (EDTA), citric acid, and aspartic acid, and mixtures thereof. The use of a chelating agent in pharmaceutical compositions is well-known to the skilled person. For convenience, reference is made to Remington: The Science and Practice of Pharmacy, 20^{th} edition, 2000.

In a further embodiment, the composition includes a stabilizer. The use of a stabilizer in pharmaceutical compositions is well-known to the skilled person. For convenience, reference is made to Remington: The Science and Practice of Pharmacy, 20th edition, 2000. More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Phann. 18:1169-1206; and Mumenthaler et al. (1994) Phann. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions may also include an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. In a further embodiment, the amino acids or amino acid analogues are used in a concentration, which is sufficient to prevent or delay aggregation of the protein.

In a further embodiment, methionine (or other sulphuric amino acids) or analogous amino acids, may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies.

In a further embodiment, the composition may include a stabilizer selected from the group of high molecular weight polymers or low molecular compounds. The stabilizer may be selected from polyethylene glycol (e.g. PEG 3350), polyvinyl alcohol (PV A), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride).

The pharmaceutical compositions may also include additional stabilizing agents, which further enhance stability of a therapeutically active protein therein. Stabilizing agents include, but are not limited to, methionine and EDTA, which protect the protein against methionine oxidation, and a nonionic surfactant, which protects the protein against aggregation associated with freeze-thawing or mechanical shearing.

In a further embodiment, the composition also includes a surfactant. The surfactant may be selected from a detergent, ethoxylated castor oil, polyglycolyzed glycerides, acetylated monoglycerides, sorbitan fatty acid esters, polyoxypropylenepolyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl serine, phosphatidyl choline, phosphatidyl ethanolamine, phosphatidyl inositol, diphosphatidyl glycerol and sphingomyelin), derivates of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyransoide), sphingoglycolipids (e.g. ceramides, gangliosides), dodecylphosphocholine, hen egg lysolecithin, fusidic acid derivatives—(e.g. sodium tauro-dihydrofusidate etc.), long-chain fatty acids and salts thereof₆-C₁₂ (e.g. oleic acid and caprylic acid), acylcarnitines and derivatives, N^{α}-acylated derivatives of lysine, arginine or histidine, or side-chain acylated derivatives of lysine or arginine, N^{α}-acylated derivatives of diproteins comprising any combination of lysine, arginine or histidine and a neutral or acidic amino acid, N^{α}-acylated derivative of a triprotein comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, amomc (alkyl-arylsulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), nonionic surfactants (e.g. Dodecyl β-D-glucopyranoside), poloxamines (e.g. Tetronic's), which are tetrafunctional block copolymers derived from sequential addition of propylene oxide and ethylene oxide to ethylenediamine, or the surfactant may be selected from the group of imidazoline derivatives, or mixtures thereof.

The use of a surfactant in pharmaceutical compositions is well-known to the skilled person. For convenience, reference is made to Remington: The Science and Practice of Pharmacy, 20^{th} edition, 2000.

It is possible that other ingredients may be present in the pharmaceutical composition. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical composition.

Pharmaceutical compositions containing a modified protein, such as e.g. a modified GH protein may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions may be administered in several dosage forms, for example, as solutions, suspensions, emulsions, microemulsions, multiple emulsion, foams, salves, pastes, plasters, ointments, tablets, coated tablets, rinses, capsules, for example, hard gelatin capsules and soft gelatin capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the protein, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behavior in lipid-water systems, polymeric micelles, multiple emulsions, self-emulsifying, self-microemulsifying, cyclodextrins and derivatives thereof, and dendrimers.

Compositions are useful in the composition of solids, semisolids, powder and solutions for pulmonary administration of a modified protein, such as e.g. a modified protein, using, for example a metered dose inhaler, dry powder inhaler and a nebulizer, all being devices well known to those skilled in the art.

### Therapeutic Uses of the Modified Proteins

To the extent that the unmodified protein is a therapeutic protein, the invention also relates to the use of the modified proteins in therapy, and in particular to pharmaceutical compositions comprising the modified proteins. Thus, as used herein, the terms “treatment” and “treating” mean the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications, The patient to be treated is preferably a mammal, in particular a human being, but it may also include animals, such as dogs, cats, cows, sheep and pigs. Nonetheless, it should be recognized that therapeutic regimens and prophylactic (preventative) regimens represents separate aspects for the uses disclosed herein and contemplated by treating physician or veterinarian.

A “therapeutically effective amount” of a modified protein as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinarian.

The methods and compositions disclosed herein provide modified proteins for use in therapy. As such, a typical parenteral dose is in the range of 10-9 mg/kg to about 100 mg/kg body weight per administration. Typical administration doses are from about 0.0000001 to about 10 mg/kg body weight per administration. The exact dose will depend on e.g. indication, medicament, frequency and mode of administration, the sex, age and general condition of the subject to be treated, the nature and the severity of the disease or condition to be treated, the desired effect of the treatment and other factors evident to the person skilled in the art. Typical dosing frequencies are twice daily, once daily, bi-daily, twice weekly, once weekly or with even longer dosing intervals. Due to the prolonged half-lives of the active compounds compared to the corresponding un-conjugated protein, dosing regimen with long dosing intervals, such as twice weekly, once weekly or with even longer dosing intervals is a particular embodiment. Many diseases are treated using more than one medicament in the treatment, either concomitantly administered or sequentially administered. It is, therefore, contemplated that the modified proteins in therapeutic methods for the treatment of one of the diseases can be used in combination with one or more other therapeutically active compound normally used in the treatment of a disease. It is also contemplated that the use of the modified protein in combination with other therapeutically active compounds normally used in the treatment of a disease in the manufacture of a medicament for that disease.

### Example 1

### In Vivo Site-Specific Protein Tagging with Diverse Amines Using an Engineered Sortase Variant

Chemoenzymatic modification of proteins is an attractive option to create highly specific conjugates for therapeutics, diagnostics, or materials in gentle, biological conditions. However, these methods often suffer from expensive specialized substrates, bulky fusion tags, low yields, and extra purification steps to achieve the desired conjugate. Staphylococcus aureus sortase A and its engineered variants are used to attach oligoglycine derivatives to the C-terminus of proteins expressed with a minimal LPXTG tag. This strategy has been used extensively for bioconjugation in vitro, and for protein protein conjugation in living cells. Here we show that an enzyme variant recently engineered for higher activity on oligoglycine has promiscuous activity that allows proteins to be tagged using a diverse array of small, commercially available amines, including several bioorthogonal functional groups. This technique can also be carried out in living Escherichia coli, enabling simple, inexpensive production of chemically functionalized proteins with no additional purification steps.

Site-specific modification of proteins is an essential technique in many scientific fields. As an example, the efficacy of antibody-drug conjugates, a therapeutic approach to cancer treatment, is enhanced by the inherent uniformity that stems from site-specific attachment of small molecule chemotherapeutics. Other in vitro protein conjugates for use in materials, imaging, diagnostics, catalysis, or devices can similarly benefit from the homogeneity of site-specific conjugation. Chemical methods for modifying proteins have historically relied on the different reactivities of specific amino acids, e.g. lysine, cysteine, and tyrosine; however, in recent years significant advances have been made to modify unique sites such as N-terminal residues₉, C-terminal residues₁₀, or glycosylated residues.

In vivo tagging of proteins, though challenging, can be used to illuminate protein localization, function, and intermolecular interactions or allow modified protein production in fewer steps than other approaches. Amber stop codon suppression using unnatural amino acids (UAAs), one of the most heavily used methods for in vivo protein labeling, can install a multitude of different functional groups in a wide variety of cell types; however, this method can be prone to decreased protein yield, truncation, and misincorporation. Enzyme fusions that employ mechanistic-based protein labeling such as SNAP-, CLIP-, TMP-, and Halo-tags feature exquisite specificity but are limited by their molecular size and expensive substrates. Numerous other chemoenzymatic methods developed to ligate orthogonal functional group adaptors allow for smaller size and greater versatility of tags.

A host of natural and engineered enzymes, such as 4′-phosphopantetheinyl transferase (Sfp), glutathione-Stransferase (GST), transglutaminase, tubulin tyrosine ligase, and phosphocholine transferase, are used to attach functional groups in vitro. Alternatively, enzymes N myristoyl transferase, biotin ligase, lipoic acid ligase, or formylglycine generating enzyme have been co-expressed with a protein of interest fused to a recognition sequence so that they attach a unique functional group in the cytoplasm.

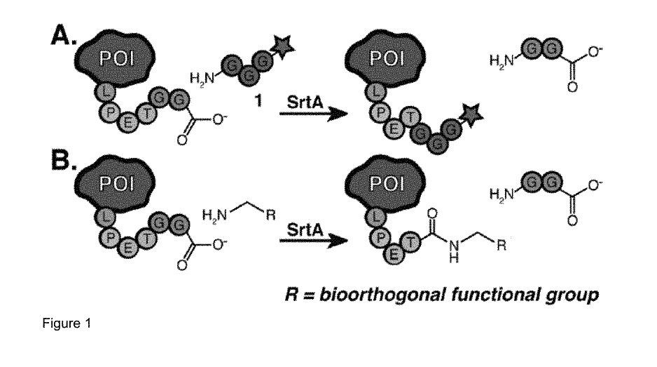

Another widely used chemoenzymatic bioconjugation approach utilizes the transpeptidase sortase A from Staphylococcus aureus to label the N- or C-terminus. This enzymatic approach is popular due to its versatility, only requiring an LPXTG recognition sequence (the “C-peptide”) and an oligo(glycine) nucleophile (the “N-peptide,” see FIG. 1A) Using this scheme, proteins have been attached to lipids, nucleic acids, polymers, drugs, inorganic materials, surfaces, thioesters, depsipeptides or other proteins. Due to the peptidic nature of the substrates, this approach has been largely limited to in vitro or cell-surface labeling, though non-natural protein-protein ligations were demonstrated in both live mammalian and E. coli cells.

Central to this latter bacterial example were two sets of mutations engineered into the sortase enzyme (termed 7M). One set [P94R/D160N/D165A/K190E/K196T], dramatically increases the activity of the enzyme. Another set, [E105K/E108A] confers calcium independence to the enzymatic activity. Each of these previous studies highlight the vast potential of sortase, but the requirement for synthetic peptide substrates limits the majority of applications to lab-scale, in vitro labeling in research groups or organizations with the resources to custom-make the desired substrates. An inexpensive, cell-permeable, commercially available bioorthogonal adaptor would greatly extend the potential of sortase for producing large scale and/or in vivo conjugates. Here we describe the use of the engineered sortase variant 7M (SrtA7M) to create bioorthogonally tagged proteins directly from E. coli culture. By simply co-expressing the sortase variant with the protein of interest and adding commercially available substrate mimics, such as 3-azido-1-propanamine (Azp) or propargylamine (FIG. 1B), we can produce large quantities of labeled protein with no extra purification steps.

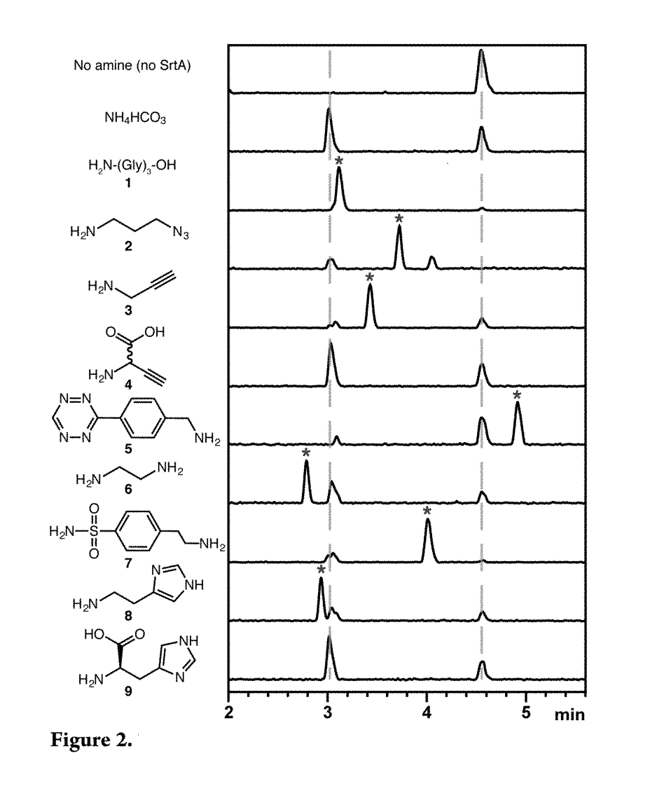

Additionally, we show this method works in vivo on a variety of protein substrates. Wild-type sortase enzymes have been used to install nonglycine nucleophiles such as aminohexoses, lysine containing sequences, some amines, and hydrazines. We hypothesized that the engineered sortase variant SrtA7M would more efficiently activate LPETG sequences with little specificity for the nucleophile. We incubated purified SrtA7M with the peptide LPETGSW, along with several potentially useful amines, and analyzed the reactions by LCMS to determine which could act as nucleophiles in the transpeptidation reaction. Six of the eight amines tested, in addition to triglycine, showed significant conversion in just two hours (FIG. 2). The reaction was tolerant to the bioorthogonally reactive groups on Azp 2, propargylglycine 3 (but not DL-propargylglycine 4), and tetrazine amine 5, in addition to the charged ethylenediamine 6 and the bulky aminoethylbenzene sulfonamide 7. Interestingly, the enzyme was able to act on histamine 8 but not histidine 9. The two amines that did not participate in the transpeptidation, 4 and 9, are branched at the alpha carbon, suggesting that the 7M variant maintains the wild-type enzyme's preference for the unbranched primary amine of oligo(glycine).

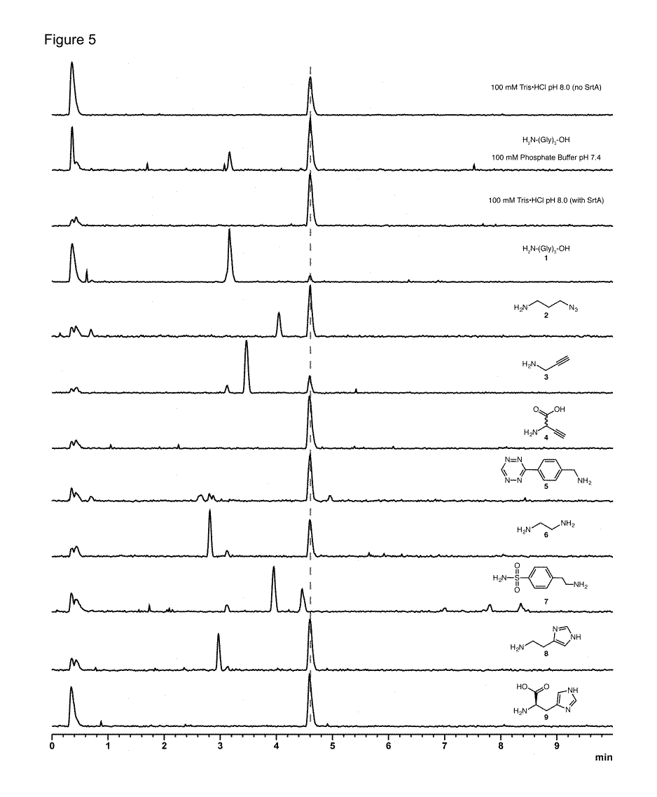

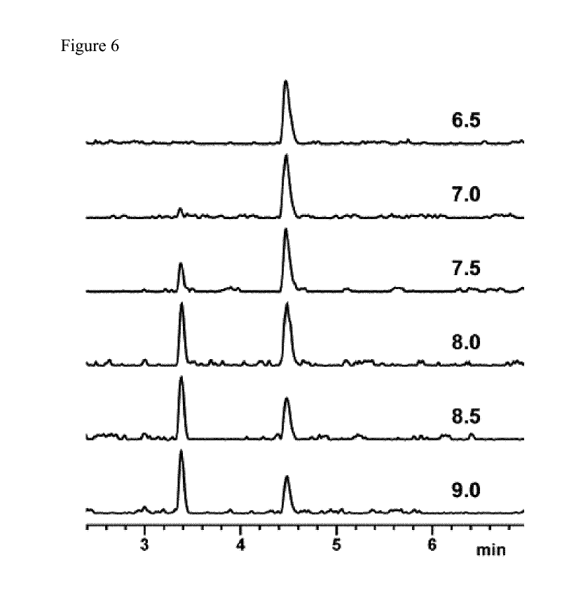

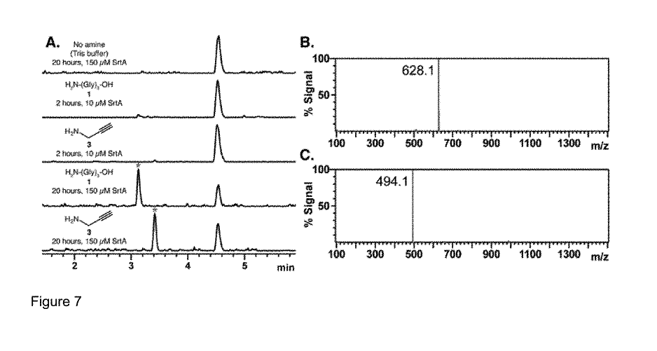

In the absence of a suitable amine, ammonia from the ammonium bicarbonate buffer was also able to add to the peptide (FIG. 2). Curiously, the enzyme was more active in this buffer; however, significant activity was also seen in Tris buffer (FIG. 5). Minimal enzymatic activity was seen in phosphate buffer. We also tested the pH-sensitivity of the reaction on propargylamine in Tris buffer and found it to work significantly better above pH 7.5 (FIG. 6). Wild-type SrtA was only able to catalyze the reaction when 15-fold more enzyme was added and incubated for 20 hours, resulting in modest conversion (FIG. 7). These data show that the enzymatic activity, but not substrate specificity, has been altered with SrtA7M compared to wild-type sortase.

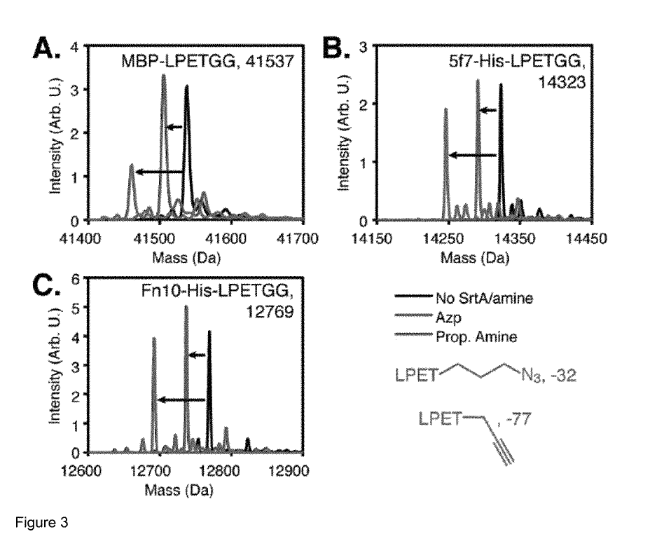

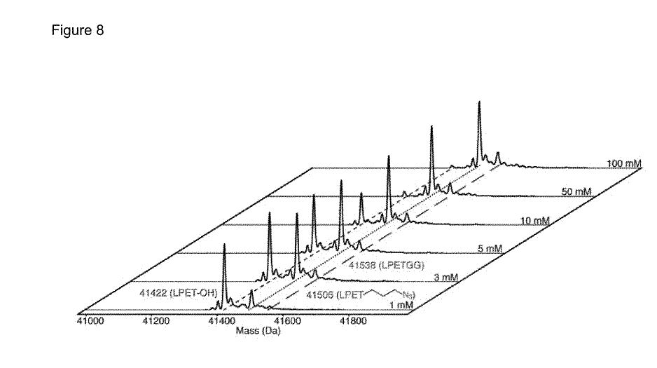

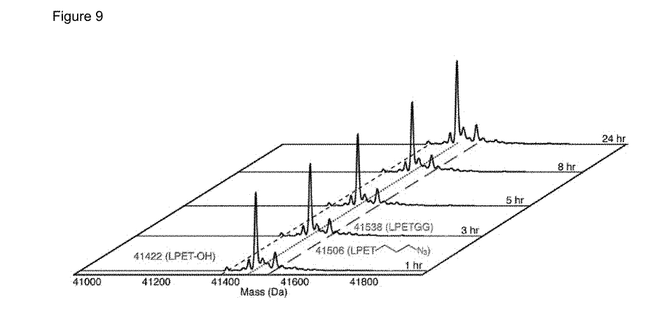

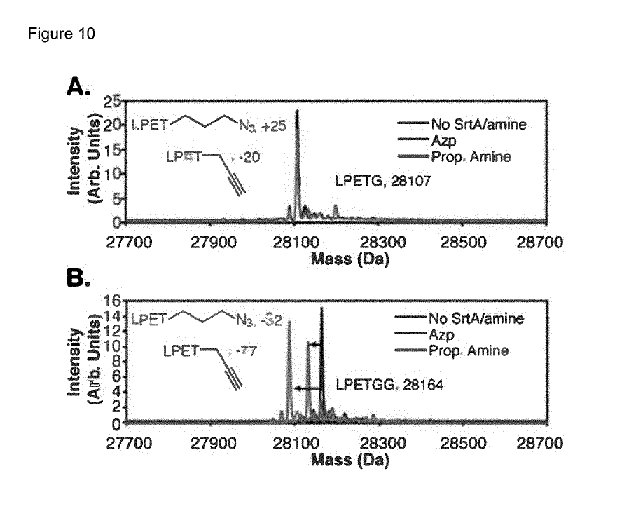

We next determined the ability of SrtA7M to modify purified proteins. We incubated several proteins containing a C terminal LPETGG sequence (srt) with 10 μM enzyme and either 100 mM Azp or propargylamine, followed by quenching with formic acid. FIG. 3 shows complete conversion of maltose binding protein (MBP-srt), anti-HER2 nanobody 5f7 (5f7-His-srt)₄₆, and the engineered fibronectin domain Fn10 (Fn10-His-srt). Additionally, little to no conversion was observed with superfolder GFP (His-sfGFP-srt) under the same conditions until the 6xHis tag was positioned as a spacer, allowing the enzyme access to the LPETGG sequence (sfGFP-His-srt; FIG. 8). To define the effective range of reaction conditions, we tested the conjugation of MBP-srt with Azp at different time points and substrate concentrations. These experiments suggested that relatively high concentrations of the amine are needed, but efficient conjugation takes place in less than 1 hour (FIGS. 9 and 10).

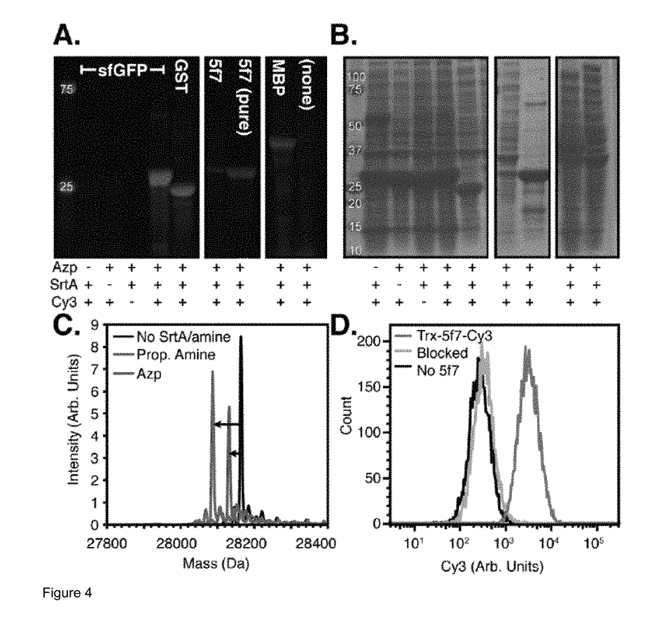

As sortase has only rarely been used in living systems, we next determined whether SrtA7M was able to install useful functional groups to proteins as they were expressed in E. coli. SrtA7M, under a rhamnose-inducible promoter, was coexpressed in BL21(DE3) cells with proteins GST-His-srt, thioredoxin-fused nanobody Trx-5f7-His-srt, or sfGFP-Hissrt under T7-inducible promoters, in the presence of Azp (FIG. 4). The azide-tagged proteins were then labeled with Cy3-dibenzocyclooctyne (Cy3-DBCO) in cell lysate and assayed by SDS-PAGE with fluorescence and Coomassie stain (FIGS. 4A and B). The LPETGG-tagged proteins were specifically conjugated only when SrtA7M was co-expressed. Trx-5f7-His-srt was only modestly expressed in E. coli but subsequent purification showed that the protein was also effectively labeled with Cy3-DBCO (FIGS. 4A and B).

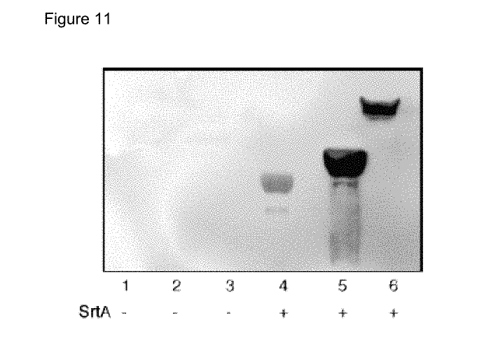

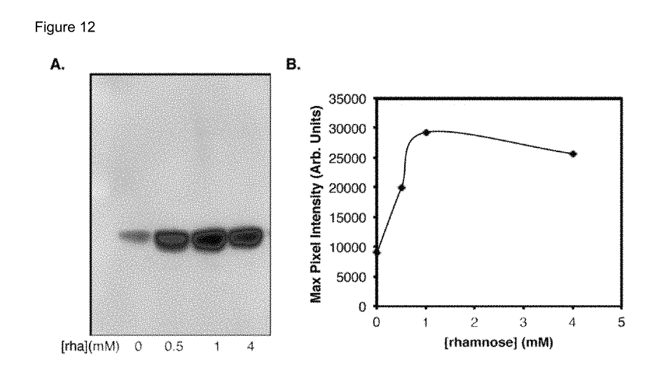

The refseq protein database contains 24 proteins containing an LPXTG sequence in E. coli BL21(DE3) proteome; however, incubating lysate of SrtA7M-expressing cells with Cy3-DBCO (FIG. 4A), or with biotin alkyne (FIG. 11) revealed minimal off-target protein conjugation with azide above background. This indicates high specificity of the sortase reaction to the protein substrate. Increased expression of sortase did not result in higher levels of protein conjugation (FIG. 12). Additionally, we co-expressed MBP-srt with SrtA7M under rhamnose- and IPTG-inducible promoters, respectively, in DH5a cells. (FIGS. 4A and B). Protein labeling with Cy3-DBCO was also successful under these conditions, indicating the sortase reaction is not dependent on cell or plasmid type.

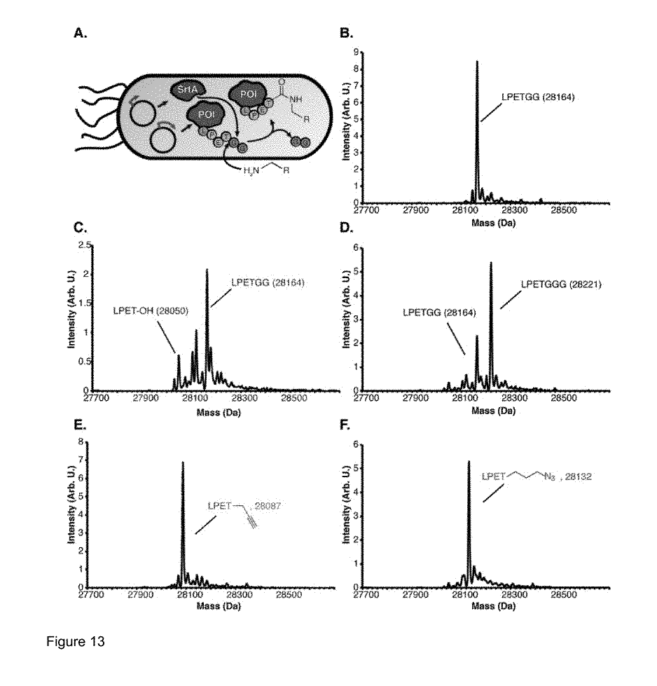

To quantify the reaction in more detail, sfGFP-His-srt was conjugated in vivo with several different substrates and purified. At the time of induction we added 25 mM Gly₃, Azp, or propargylamine to the cultures and incubated for 24 hr at 30° C. After expression and conjugation, the proteins were purified with Ni₂₊/NTA resin and analyzed by LCMS. Incubation with Gly₃ resulted in partial conjugation of the LPETGG tag, while incubation with Azp or propargylamine resulted in complete conjugation of the protein (FIG. 4C and FIG. 13). In the absence of amine, several other uncharacterized peaks were present in the purified protein, suggesting that SrtA7M is able to conjugate intracellular E. coli metabolites or media components, in addition to hydrolysis at threonine (FIG. 13).

Finally, we demonstrated the utility of our simple labeling method by making a Cy3-tagged HER2-binding imaging agent in a single expression and purification step. After expression and Azp conjugation, Trx-5f7 was bound to Ni2+/NTA resin and labeled on-column with Cy3-DBCO. This probe was effective in detecting HER2 expression on SK-BR-3 breast cancer cells (FIG. 4D). Site-specific modification with alternate nucleophiles has been demonstrated in other sortase-mediated protein labeling experiments, but only in specialized cases. Similar thioester-trapping techniques have been performed using intein domains and butelase, but not in living cells. Here we show for the first time a general, high-yield protein modification strategy using inexpensive bioorthogonal reagents. In addition, we show that this strategy is effective in living E. coli cells, paving the way for further engineering of specific, highly active enzymes for in vivo protein experiments. Future engineering of sortase to repress proteolytic activity and activity on other cellular amines will improve performance and specificity for amine nucleophiles.

Reagents. Chemicals from Sigma unless otherwise noted. 10× Tris Buffered Saline (TBS), Tris Base and Lysogeny Broth were purchased from Thermo-Fisher Scientific (Pittsburgh, Pa.). Phusion polymerase, restriction enzymes and T4 DNA ligase were purchased from New England Biolabs (Ipswich, Mass.). Ultrapure Agarose, Terrific Broth and Deoxyribonuclease I were purchased from Invitrogen (Grand Island, N.Y.). Isopropyl-β-D-thiogalactopyranoside (IPTG) and lysozyme were purchased from Gold Biotechnology (St. Louis, Mo.), and Neutravidin-HRP, Bacterial Protein Extraction Reagent (BPER) and SuperSignal West Femto Maximum Sensitivity Substrate were from Thermo Scientific (Waltham, Mass.). All experiments were performed using deionized, filtered water from a MilliQ system (Millipore, Billerica, Mass.)

General Equipment. PCR and Golden Gate cloning were performed using a T100 Thermocycler (Biorad, Hercules, Calif.). UV/Vis spectroscopy was performed using a Nanodrop 2000 (Thermo Fisher Scientific, Pittsburgh, Pa.). DNA electrophoresis was performed using a BioRad Mini Sub Cell GT and Powerpac Basic (BioRad, Hercules, Calif.). Gels were imaged with Ethidium Bromide on a Proteinsimple Red Imager (Protein Simple, San Jose, Calif.). Protein electrophoresis was performed using ExpressPlus PAGE Gels (12% or 4-20% gradient) (Genscript, Piscataway, N.J.) in an XCell Novex Mini Cell (Invitrogen, Grand Island, N.Y.) in MOPS/SDS buffer according to the manufacturer's instructions. Western Blots were performed using the iBlot system using nitrocellulose membranes (Invitrogen, Grand Island, N.Y.), blocked with 5% milk in TBS+0.5% Tween (TBST, Sigma Aldrich, St. Louis, Mo.). Fluorescent imaging of SDS-PAGE gels was performed with a Typhoon 9500 (GE Healthcare, Pittsburgh, Pa.).

Peptide Synthesis. Solid phase peptide synthesis (SPPS) was performed on a CS Bio CS336 instrument (Menlo Park, Calif.), using 9-fluorenylmethyloxycarbonyl (Fmoc)-protected amino acids and Rink amide resin (CS Bio). Fmoc groups were removed with 20% piperidine in N,N-dimethylformamide (DMF). Amino acid coupling was performed using 1-hydroxybenzotriazole/diisopropylcarbodiimide (HOBT/DIC) chemistry in DMF. Peptides were synthesized with a 2 hour coupling/washing/deprotection cycle. Side-chain deprotection and resin cleavage was performed by suspending the resin in 10 ml of a 94:2.5:1:2.5 (v/v) mixture of trifluoroacetic acid (TFA): 1,2-ethanedithiol:triisopropylsilane:water for 2 hr at room temperature open to air. Resin was filtered off, and crude peptides were precipitated with 10 volumes of diethyl ether cooled to −80° C., isolated by filtration, and washed with diethyl ether. The peptide was then resuspended in water with 0.1% TFA and purified by preparative reverse-phase HPLC on Varian Prostar instrument using a Vydac C18 column, and eluted with a linear gradient of 90% acetonitrile with 0.1% TFA over 20 min. Chromatography was followed by UV absorbance at 220 nm, and fractions containing peptide were collected, frozen in dry ice, and lyophilized.

Mass Spectrometry. Peptide Liquid Chromatography/Mass spectrometry (LC/MS) was performed using a Shimadzu 2020 Liquid Chromatograph Mass Spectrometer (Shimadzu). Peptide samples were run on a Synergi 4u Hydro-RP 80 Å 30×2.0 mm column (Phenomenex). Protein samples were analyzed using an Agilent 1200 series HPLC in line with an Agilent 6224 TOF mass spectrometer with a Turbospray ion source. Protein samples were run on a Poroshell 300SB-C18 column (Agilent Technologies). Protein mass reconstruction was performed with Mass Hunter software (Agilent, USA).

Molecular Biology. Cloning was performed using standard molecular biology techniques. Plasmid pET30b-SrtA5M was obtained as a generous gift from Brian McNaughton (Colorado State University). Constructs cloned into pTrc99a were amplified using 5′ NcoI site and a 3′ BamHI site. Constructs in pRha were cloned into a custom-made rhamnose inducible vector (pRhaGG, DNA2.0) using Golden Gate cloning with 5′ and 3′ BsaI sites. Constructs in pET22b were cloned using Golden Gate cloning by amplifying the pET22b backbone with primers 152 and 153, installing BsaI restriction sites. All plasmids were transformed into chemically competent DH5α cells and plated on appropriate antibiotics. Sortase 7M (SrtA7M) in pTrc99a was generated from pTrc99a-SrtA5M by quick change mutagenesis using primers 41 and 42.

| Primers | Sequence | Description |
|---|---|---|
| JEG28_malE_R | 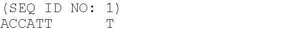 | Cloning MBP into pRhaGG |
| JEG31_malEdF | 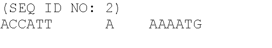 | |
| JEG41 | (SEQ ID NO: 3) | Quikchange 5M to 7M |
| | GAGGTGTAAGCTTTGCAAAAGAAAATCAATC | |
| | ACTAGATGATCAAAATATTTC | |
| JEG42 | (SEQ ID NO: 4) | |
| | GAAATATTTTGATCATCTAGTGATTGATTTTCT | |
| | TTTGCAAAGCTTACACCTC | |
| JEG60_malE_Srt_R | 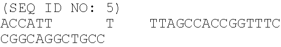 | Cloning MBP-Srt into pRhaGG |
| JEG92_SrtPT_F | 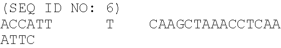 | Cloning SrtA variants into pTrc99a |
| JEG93_SrtPR_R | 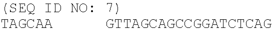 | |
| JEG127_GFP_F | 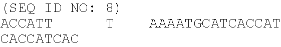 | Cloning His-sfGFP-srt |
| JEG128_GFP_R | 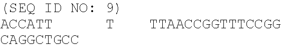 | |
| JEG144_SGSH_F | 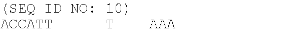 | Cloning sfGFP-His-Srt into pET22 |
| JEG145_SGSH_R | 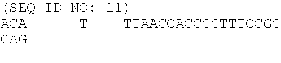 | |
| JEG146_Srt_Rh_F | 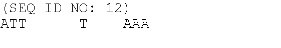 | Cloning SrtA into pRhaGG |
| JEG147_Srt_Rh_R | 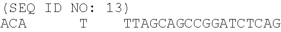 | |
| JEG152_p22_F | 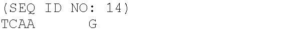 | Reverse PCR pET22b for Golden Gate |
| JEG153_p22_R | 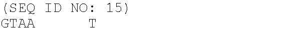 | |
| JEG156_Nan_Rh_F | 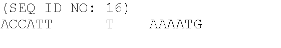 | Cloning Nanobody 5f7- His-Srt into pET22 |
| JEG157_Nan_Rh_R | 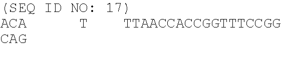 | |
| JEG160_Fn10_F | 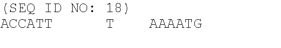 | Cloning Fibronectin FN10-His-Srt into pET22 |
| JEG161_FnHS_R | 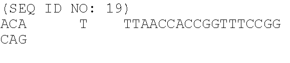 | |
| JEG162_GST_F | 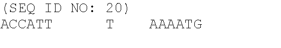 | Cloning Glutathione-S- transferase-His-Srt into pET22 |
| JEG163_GSTHS_R | 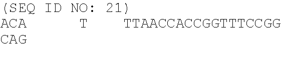 | |
| JEG129_Trx_F | 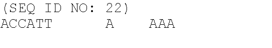 | Installing thioredoxin tag on to Nanobody 5f7 in pET22 |
| JEG130_Trx_R | 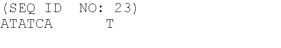 | |
| JEG165_NB_Trx | 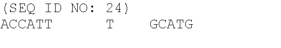 | Cloning Nanobody fusion Trx-5f7-His-srt |

Sortase variants. Sortase 5M and 7M and wild-type SrtA Δ59 were expressed and purified in E. coli. Plasmid pET30b-SrtA5M was transformed into BL21(DE3)pLysS cells (Invitrogen). Colonies grown on LB agar containing 50 μg/ml kanamycin and 34 μg/ml chloramphenicol were inoculated into 5 ml LB media containing the same antibiotics and grown overnight at 37° C. Cells were then subcultured 1/100 into 500 ml of TB media containing 100 μg/ml kanamycin and 34 μg/ml chloramphenicol and grown until cells reached mid-log phase (OD ˜0.4). Expression of the sortase variant was induced with 1 mM IPTG. Cells were allowed to express protein overnight at 37° C. Cells were then harvested by centrifugation at 4000×g and resuspended in BPER (Pierce) with 0.6 mg/ml lysozyme by vortexing. Deoxyribonuclease was added and suspension was incubated at room temperature for 10 min with frequent vortexing. Insoluble material was pelleted at 15000×g for 15 min. The soluble fraction was applied to 1 ml of Ni₂₊/NTA resin preequilibrated with wash buffer (50 mM TrisHCl, 300 mM NaCl, 20 mM imidazole pH 7.4). The resin was washed with another 60 ml of wash buffer. Proteins were eluted with elution buffer (50 mM TrisHCl, 300 mM NaCl, 300 mM imidazole pH 7.4). The protein solution was dialyzed into 1×TBS (Fisher) using Slide-A-Lyzer dialysis cassettes (Pierce), concentrated using a 10,000 Da MWCO centrifugal filter (Millipore, Billerica, Mass.), and stored at 4° C. Wild-type SrtA Δ59 was amplified from pGBMCS-SortA (Addgene) and cloned into pET20b using SapI Golden Gate cloning, and expressed similarly, with the addition of 10% glycerol to the purification buffers.

GFP-His-Srt, Nanobody-His-Srt, Fibronectin-His-Srt, Glutathione-S-Transferase-His-Srt.

Plasmid pET22 containing the gene of interest was transformed into BL21(DE3) cells, which were then plated on LB+100 μg/ml ampicillin. Colonies were inoculated into LB+Amp and grown overnight, then subcultured 1/100 into 100-1000 ml LB+Amp, grown to mid-log phase, and induced with 1 mM (final) IPTG and cooled to 30° C. Protein was expressed overnight at 30° C. Cells were then harvested by centrifugation at 4000×g and resuspended in BPER (Pierce) with 0.6 mg/ml lysozyme by vortexing. Deoxyribonuclease was added and suspension was incubated at room temperature for 10 min with frequent vortexing. Insoluble material was pelleted at 15000×g for 15 min. The soluble fraction was applied to 1 ml of Ni₂₊/NTA resin preequilibrated with wash buffer (50 mM TrisHCl, 300 mM NaCl, 20 mM imidazole pH 7.4). The resin was washed with another 60 ml of wash buffer. Proteins were eluted with elution buffer (50 mM TrisHCl, 300 mM NaCl, 300 mM imidazole pH 7.4). The protein solution was dialyzed into 1×TBS (Fisher) using Slide-A-Lyzer dialysis cassettes (Pierce), concentrated using a 10,000 Da MWCO centrifugal filter (Millipore, Billerica, Mass.), and stored at 4° C.

Maltose Binding Protein-Srt (MBP).

MBP without its N-terminal periplasmic localization tag, containing a C-terminal LPETGG was expressed from pRha in DH5a. The plasmid was transformed into the cells, which were then plated on LB+50 μg/ml kanamycin. Colonies were inoculated into LB+Kan and grown overnight, then subcultured 1/100 into 100 ml of LB+Kan, grown to mid-log phase, and induced with 4 mM (final) rhamnose. Protein was expressed overnight at 37° C. The next day cells were harvested and lysed with BPER+0.6 mg/ml lysozyme, followed by digestion with 2 units of DNase I. The lysate was clarified by centrifugation at 14,000×g, and the supernatant was applied to a S5 1 ml MBP-Trap column (GE Healthcare). The bound protein was washed with 10 ml of Buffer A (100 mM Tris, 300 mM NaCl, 1 mM EDTA, pH 7.2) and then eluted with 5 ml of Buffer A+10 mM maltose. The protein was concentrated and buffer exchanged into 10 mM phosphate buffer using 10 kDa MWCO centrifugal filters.

In vitro conjugation of peptides. Synthetic LPETGSW peptide was resuspended to 20 mM in water. Stock solutions (50 mM) of each amine were made in 50 mM acetic acid. Reactions consisting of 1 mM peptide, 10 mM amine, 100 mM ammonium bicarbonate, and 20 μM SrtA7M (10 μl final volume) were incubated at 37° C. for 2 hr with occasional mixing. Reactions were quenched with the addition of 9 volumes of 0.2% formic acid and analyzed by LC/ESI-Quadrupole MS. The pH dependence of the enzyme was analyzed as above, except in 100 mM Tris.HCl at the pH described. Reactions were allowed to proceed at 37° C. for 2 hours, followed by formic acid quenching and LC/MS as described. Peptide labeling with wild-type SrtA Δ59 was performed in 25 mM Tris pH 7.5, 150 mM NaCl, 10 mM CaCl2. Enzyme (10 μM or 150 μM final) was added to buffer containing 10 mM amine of interest and 1 mM peptide. Reactions were allowed to proceed for 2 or 20 hours, followed by quenching and LC/MS analysis as described.

In vitro conjugation of proteins. Purified MBP-srt, His-sfGFP-srt, Nanobody-His-srt, Glutathione-S-transferase-His-srt, Fibronectin-His-srt, and sfGFP-His-srt were labeled after purification in 100 mM ammonium bicarbonate buffer pH 7.8. MBP-Srt, HissfGFP-Srt, sfGFP-His-srt, and Glutathione-S-transferase-His-Srt were labeled at a final concentration of 100 μM, while Nanobody-His-Srt, and Fibronectin-His-Srt were labeled at final concentrations of 80 μM and 50 μM, respectively, due to solubility issues. Concentrated Azp and propargylamine were each mixed with 1 M acetic acid to a final concentration of 1 M and added to the reaction at a final concentration of 100 mM. The reaction was initiated with the addition of SrtA7M and incubated at 37° C. for 8 hr. Each reaction was then quenched with 9 volumes of 0.2% formic acid and analyzed by LC/ESI-TOF-MS.

Coexpression, conjugation, and purification of MBP variants. MBP-srt in pRha and the SrtA 7M in pTrc99a were cotransformed into DH5α cells. Colonies grown on LB agar with 50 μg/ml kanamycin and 100 μg/ml ampicillin were grown inoculated into 2 ml LB media with the same antibiotics and grown overnight at 37° C. Overnight cultures were subcultured 1/100 into 2-25 ml of the desired media with the appropriate antibiotics, supplemented with 5 mM CaCl2 and 5 mM MgCl₂. After 2 hr growth at 37° C., expression of MBP was induced with 4 mM rhamnose. Sortase was not induced. At this time the desired concentration of Azp was added. Background expression of sortase was sufficient to see significant conjugation. Cells were allowed to express and modify the MBP for 20 hr. 500 μl of the cells were harvested by centrifugation and washed three times with TBS. Cells were then resuspended in 100 μl BPER with 0.6 mg/ml lysozyme and 0.5 units of DNase. Lysate was then clarified by centrifugation at 15,000×g at 4° C. for 10 min. Clarified lysate was then analyzed directly or carried through to purification. S6 MBP variants were purified using a 1 ml MBP-trap column (GE Healthcare Pittsburgh, Pa.). The lysate was applied to the column preequilibrated with 2×TBS with 1 mM EDTA. The bound protein was washed with 10 ml of the same buffer, followed by elution with 5 ml of the same buffer with 10 mM maltose. Protein was concentrated and desalted using 30 kDa MWCO spin filters (Millipore, Billerica, Mass.).

Coexpression, conjugation, and purification of Trx-Nanobody-His-srt, Glutathione-S-transferase-His-srt, and sfGFP-His-srt. The desired target protein in pET22b was cotransformed with pRha-SrtA7M into BL21(DE3) cells (Invitrogen). Colonies grown on LB agar with 50 μg/ml kanamycin and 100 μg/ml ampicillin were inoculated into 2 ml LB media with the same antibiotics and 1 mM rhamnose and grown overnight at 37° C. Overnight cultures were subcultured 1/100 into 2-25 ml of LB with the appropriate antibiotics, supplemented with 5 mM CaCl2, 5 mM MgCl₂, and 1 mM rhamnose. After 2 hr growth at 37° C., expression of the target protein was induced with 1 mM (final) IPTG and the desired concentration of Azp or propargylamine was added and the temperature was lowered to 30° C. Cells were allowed to express and modify the desired protein for 20 hr. 500 μl of the cells were harvested by centrifugation and washed three times with TBS. Cells were then resuspended in 100 μl BPER with 0.6 mg/ml lysozyme and 0.5 units of DNase and 50 mM iodoacetamide. Lysate was then clarified by centrifugation at 15,000×g at 4° C. for 10 min. Clarified lysate was then analyzed directly or carried through to purification. Azp- or propargylamine-conjugated, His-tagged proteins were purified as described above.

Analysis of Azp conjugation by fluorescent labeling. Lysate from in vivo experiments (30 μl) was incubated with 500 μM Cy3-DBCO (Sigma) overnight at room temperature. The following day, any insoluble material was pelleted by centrifugation at 15000×g, and 15 μl of the remaining lysate was run on a 4-20% gradient SDS-PAGE gel. The gel was imaged for fluorescence followed by staining with Coomassie.

Analysis of Azp conjugation by western blot. Lysate from in vivo experiments was directly labeled with Sulfo-dibenzocyclooctyne (DIBAC)-biotin (Sigma). The reaction was initiated by adding the biotin-DIBAC (0.6 μl, 50 mM in DMF) directly to lysate (30 μl) containing Azp-labeled proteins. The solution was incubated at room temperature for 3 hr with occasional mixing. Then 12 μl of 4×LDS buffer and 4 μl of 10× reducing agent (Invitrogen) were added to each sample, followed by heating at 95° C. for 10 min. The samples were cooled and run on a 12% SDS-PAGE gel (Genscript) at 125 V for 65 min, followed by transfer to a nitrocellulose membrane using the iBlot system (Invitrogen). The membrane was blocked with 5% milk in TBST for 1 hr. The membrane was then stained with a 1/1000 dilution of neutravidin-HRP (Thermo-Pierce) in 5% milk in TBST for 1 hr at room temperature. Unbound neutravidin-HRP was washed from the blot three times with TBST for 5 min each. The blot was then imaged using SuperSignal West Femto Maximum Sensitivity Substrate (Thermo-Pierce).

Coexpression, conjugation, and purification of Trx-Nanobody-His-srt for HER2-binding assays. Thioredoxin-fused nanobody 5f7 (in pET22) was coexpressed with S7 SrtA7M (in pRha) as described above in 50 ml of LB with 100 μg/ml ampicillin, 50 μg/ml kanamycin, 5 mM MgCl₂, 5 mM CaCl2, and 1 mM rhamnose. An overnight culture was subcultured 1/100 and grown at 37° C. for 2 hours followed by addition of IPTG to 1 mM and Azp to 25 mM (pH adjusted to 7 with acetic acid). The cells were allowed to express and label for 24 hours at 30° C. Cells were then harvested and lysed with 5 ml BPER+1 mg/ml lysozyme and 0.5 units of Dnase. The lysate was clarified by centrifugation at 14,000×g for 10 minutes and applied to 0.5 ml Ni₂₊/NTA resin. The resin was washed with 20 ml of wash buffer (50 mM TrisHCl, 300 mM NaCl, 20 mM imidazole pH 7.4). 1 ml of 100 μM Cy3-DBCO was then added to the resin, transferred to a 15 ml falcon tube, followed by 1 ml wash buffer to transfer any remaining resin. The mixture allowed to react overnight at room temperature with agitation. The resin was transferred back to the column and washed with an additional 20 ml wash buffer. The bright red protein was then eluted with 4 ml wash buffer with 300 mM imidazole. This was concentrated and buffer exchanged using a 10 kDa MWCO centrifugal filter and used without further purification. Protein concentration was obtained using the “dyes and labels” setting on a Nanodrop.

Purification of unlabeled Trx-5f7. pET22 containing the gene for Trx-5f7 was transformed into Shuffle T7 cells (New England Biolabs) and plated on LB with 100 μg/ml ampicillin. A single colony was used to inoculate liquid LB+Amp and grown overnight at 37° C. The overnight was subcultured 1/100 in 50 ml of LB with 100 μg/ml ampicillin, 5 mM MgCl₂, 5 mM CaCl₂, grown for 2 hours and induced with 1 mM IPTG. The cells were allowed to express and label for 24 hours at 30° C. Cells were then harvested and lysed with 5 ml BPER+1 mg/ml lysozyme and 0.5 units of Dnase. The lysate was clarified by centrifugation at 14,000×g for 10 minutes and applied to 0.5 ml Ni₂₊/NTA resin. The resin was washed with 40 ml of wash buffer (50 mM TrisHCl, 300 mM NaCl, 20 mM imidazole pH 7.4). The protein was then eluted with 4 ml wash buffer with 300 mM imidazole, concentrated and buffer exchanged using a 10 kDa MWCO centrifugal filter and used without further purification. Protein concentration was obtained using the Protein A280 setting on a Nanodrop.

Flow cytometry analysis of labeled nanobody binding. SK-BR-3 cells (ATCC) were cultured in a T75 flask using Mccoy's 5a media supplemented with 10% FBS. Cells at 80% confluence were treated with 3 ml of 0.25% trypsin/EDTA for 3 minutes, followed by quenching with 8 ml fresh media. Cells were washed three times with Hyclone PBS by centrifugation at 1000×g for 3 minutes and resuspension in 8 ml PBS, and finally resuspended in Hyclone PBS with 0.1% BSA to a final concentration of 400,000 cells/ml and kept on ice for the remainder of the protocol. A 100 μl solution of this, containing 40,000 cells, was treated with either 100 nM labeled Trx-5f7 alone, or pretreated (20 minutes) with 10 μM unlabeled Trx-5f7. Nanobody-cell mixtures were incubated on ice for 20 minutes with occasional agitation. Cells were then washed twice and pelleted. Cells were then resuspended in PBS+0.1% BSA (150 μl) immediately before analysis by flow cytometry. Histograms were generated using FlowJo.

### Example 2

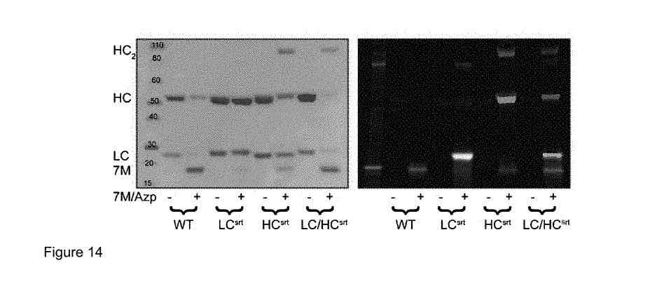

Both localization and drug to antibody ratio (“DAR”) are known to influence the efficacy of antibody-drug conjugates. Sortase-based modification is useful for attaching cargo to antibodies in high yield with site-specificity, as demonstrated by the attachment of Cy3 specifically to the light chain, heavy chain, or both chains. Trastuzumab light and heavy chains were separately cloned into plasmid pCEP4 with and without C-terminal sortase recognition sequence with a Gly-Ser spacer (GSLPETGG). Combinations of these proteins were expressed from HEK293F cell culture and purified using protein A resin: WT (no recognition sequence), LC^{srt} (recognition sequence on light chain only), HC^{srt} (recognition sequence on heavy chain only), and LC/HC^{srt} (recognition sequence on both chains). The purified antibodies were incubated with 20 μM SrtA7M in 100 mM ammonium bicarbonate buffer in the presence of 100 mM 11-Azido-3,6,9-trioxaundecan-1-amine (Sigma 17758) overnight at 37° C. The protein was buffer exchanged into Tris-Buffered Saline (TBS) using 10 kDa MWCO spin filters (Millipore). DBCO-Cy3 (Sigma 777366) was then added to 100 μM, and the mixture was incubated at room temperature overnight. Control proteins not treated with sortase were similarly incubated with DBCO-Cy3. NuPage 4×LDS sample buffer (ThermoFisher NP0007) and 10× reducing agent (NP0009) were added to 1× and the samples were heated to 70° C. for 10 minutes. Proteins were then analyzed by SDS-PAGE and first imaged on a Typhoon 9500 imager (GE Healthcare) to visualize Cy3-conjugated protein followed by staining with Coomassie to visualize total protein. FIG. 14 shows specific labeling of the desired chain with minimal conjugation to proteins lacking the sortase recognition sequence. Curiously, the sortase in the reaction was also labeled with Cy3-DBCO, possibly through a thiol-yne reaction due to extended incubation with the strained alkyne dye. Dimerization of heavy chains with the recognition sequence was also observed.

### Example 3

### Dual Display of Proteins on the Yeast Cell Surface Simplifies Quantification of Binding Interactions and Enzymatic Bioconjugation Reactions

Yeast surface display, a well-established technology for protein analysis and engineering, involves expressing a protein of interest as a genetic fusion to either the N- or C-terminus of the yeast Aga2p mating protein. Historically, yeast-displayed protein variants are flanked by peptide epitope tags that enable flow cytometric measurement of construct expression using fluorescent primary or secondary antibodies. Here, we built upon this approach to develop a new yeast display strategy that comprises fusion of two different proteins to Aga2p, one to the N-terminus and one to the C-terminus. Using this approach allows an antibody fragment, ligand, or receptor to be directly coupled to expression of a fluorescent protein readout, eliminating the need for antibody-staining of epitope tags to quantify yeast protein expression levels. We show that this system simplifies quantification of protein-protein binding interactions measured on the yeast cell surface. Moreover, we showed that this system facilitates co-expression of a bioconjugation enzyme and its corresponding peptide substrate on the same Aga2p construct, enabling enzyme expression and catalytic activity to be measured on the surface of yeast.

Recombinant proteins are expressed and tethered on the surface of cells to enable measurement of biophysical and biochemical properties in a paralleled, high-throughput manner. Since the first surface expression system was introduced on bacteriophage in mid-1980s, a variety of techniques have been developed to display proteins on the surface of bacteria, yeast, insect, or mammalian cells. In these platforms, each individual cell harbors a genetically-encoded protein variant of interest fused to a cell surface anchor protein. The protein of interest becomes accessible to the extracellular space after the fusion construct, directed via signal sequence, is transported to the cell wall or outer membrane. Using these methods in conjunction with plasmids encoding diverse libraries of protein variants, multiple copies of a unique protein variant are displayed on the surface of each individual cell. The libraries are screened to isolate cells that express proteins with a desired phenotype, and selected protein variants are identified by sequencing the corresponding genetic material recovered from the cells. This genotype-to-phenotype linkage is central to the application of the surface display techniques in combinatorial protein engineering.

Over the past two decades, yeast surface display has been extensively used to analyze and engineer proteins with increased binding affinity to a target of interest, thermal stability, or catalytic properties. Compared to the other cell surface display platforms, yeast surface display has collective advantages such as eukaryotic post-translational modifications, the ease of cell culture and genetic modification, and the compatibility with flow cytometric analysis. In the display platform pioneered by Boder and Wttrup in 1997, yeast cells are transformed with a plasmid encoding a protein of interest genetically fused to the a-agglutinin mating protein Aga2p subunit. After translation, the Aga2p subunit is covalently bound to an integrated Aga1p subunit via two disulfide bonds, processed through the yeast secretory machinery, and transported to the exterior of the yeast cell surface, where Aga1p is covalently anchored to the cell wall (FIG. 1A). The surface-displayed protein of interest is flanked by peptide epitope tags (i.e. c-Myc and hemagglutinin antigen (HA) tags) which enable quantification of fusion protein expression levels on each cell using fluorescent antibodies and analysis by flow cytometry. This feature allows for discrimination of protein variants with only a 2-fold difference in affinity by normalizing binding signal with expression levels on the yeast cell surface.

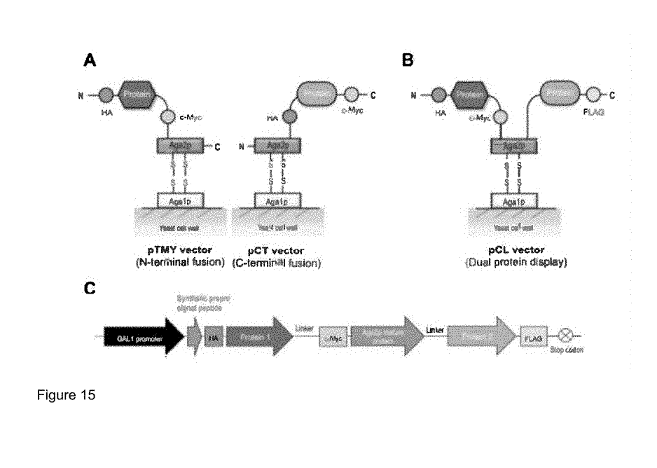

Conventional yeast surface display vectors are designed to display a protein of interest fused to either the N- or C-terminus of Aga2p (FIG. 15A). Alternatively, homodimeric and heterodimeric proteins have been displayed on yeast using a single vector containing two GAL1 promoters or a bidirectional GAL1-10 promoter. Using these strategies, the heavy and light chains of an anti-streptavidin Fab or class II MHC α and β chains have been displayed as fusions on yeast surface. As another example, homo-oligomeric streptavidin was functionally expressed and assembled using two yeast display vectors. Such multi-protein display can also aid in enzyme engineering, where substrate channeling imparts significant kinetic advantages.

Here, we introduce a simplified yeast display strategy that utilizes both the N- and C-termini of Aga2p to display two heterologous proteins as part of one fusion protein (FIG. 15B). We show that a number of different proteins can be anchored in this manner and retain their functional activity. In one demonstration, dual expression of a fluorescent protein along with a ligand, receptor, or antibody fragment simplifies quantification of protein expression by eliminating the need for antibody staining of epitope tags. This approach saves time and cost, allowing streamlined determination of equilibrium binding constants compared to conventional yeast surface display. In a second demonstration, we show that the dual expression of the bioconjugation enzyme Staphylococcus aureus sortase A and its corresponding peptide substrate as part of the same Aga2p construct enables measurement of catalytic activity on a non-natural substrate. This approach is simple and more generalizable compared to a previously reported method.

### Materials and Methods

Strains and reagents. Chemical competent TOP10 E. coli cells were used to clone, propagate, and store the plasmids through cultures in LB media containing 100 μg/ml ampicillin for selection. Saccharomyces cerevisiae strain EBY100 was used for yeast surface display throughout this study. Plasmids were transformed into yeast by homologous recombination using a Gene Pulser Xcell electroporation system (Bio-Rad). Transformed yeast were grown in SD-CAA media (20 g dextrose; 6.7 g Difco yeast nitrogen base; 5 g Bacto casamino acids; 5.4 g Na₂HPO₄; 8.56 g NaH₂PO₄.H₂O; dissolved in deionized H2O to a volume of 1 L) and induced to express Aga2p fusion proteins on their surface through a galactose-inducible promoter by culturing in SG-CAA media (prepared as SD-CAA except using 20 g galactose substituted for dextrose). For the sortase bioconjugation reaction, a 10 M stock of 3-azido-1-propanamine (Azp) (Sigma, 762016) was diluted to 1 M in 1 M acetic acid immediately before use. Sulfo-dibenzocyclooctyne-biotin conjugate (Sigma, 760706) was stored as a 50 mM stock solution in dimethylformamide at −20° C.

Construction of plasmids. The pCL vector was generally designed as shown in FIG. 15C and created by rebuilding the pTMY vector which displays the protein-of-interest, HA, and c-Myc epitope tags as a fusion to the N-terminus of Aga2p. The recombinant yeast-codon-optimized enhanced GFP with mutations S65G and S72A (termed yEGFP throughout this study) was kindly provided by Prof. Eric Shusta at University of Wisconsin-Madison. The upstream region of pTMY (N-terminal to the Aga2p mature protein) was preserved (FIG. 15C) and includes a GAL1 promoter, followed by a synthetic α-factor prepro signal peptide, a KR (Lys-Arg) KEX2 cleavage sequence and an EA (Glu-Ala) peptide spacer. In addition to a flexible (Gly₄Ser)₃ linker incorporated into pTMY upstream of the Aga2p protein, another (Gly₄Ser)₃ linker was introduced downstream of Aga2p to provide spatial degrees of freedom for proteins tethered at the N- and C-termini of Aga2p. To prevent homologous recombination within the plasmid DNA, nucleotide sequences were varied in the new (Gly₄Ser)₃ linker. The downstream region of this new linker was modified in each pCL vector and the epitope tags such as HA, c-Myc, or FLAG tags were inserted, removed, or relocated according to the design of each recombinant pCL vector.

Binding assays. For binding assays of the selected model proteins, yeast cells were transformed with the GFP-co-expressing pCL plasmids (pCL-nGFP-Aga2p-D1.3, pCL-nGFP-Aga2p-Axl, and pCL-NK1-Aga2p-cGFP) and the corresponding pCT or pTMY plasmids (pCT-D1.3, pCT-Axl, and pTMY-NK1). After growth in SD-CAA media at 30° C. to an OD₆₀₀=3-6, yeast cells were centrifuged and resuspended to a final OD₆₀₀ of 1 in SG-CAA media followed by 24 h incubation at 20° C. for induction of protein expression. Target binding affinities of each model protein on the pCL or pCT/pTMY plasmids were measured by incubating induced yeast cells with varying concentrations of target protein in PBSA (phosphate-buffered saline+1 mg/ml BSA) for 6-17 h at room temperature. Reaction volumes and time were empirically determined to minimize ligand depletion and to ensure equilibrium was reached. After incubation, yeast cells expressing proteins using the pCL-GFP plasmids were stained with a fluorescently-labeled secondary antibody against the target protein to measure binding signals. For yeast harboring pCT or pTMY plasmids, cells were first stained with a primary antibody that binds to an epitope tag (to quantify protein expression levels) and then labeled with secondary antibodies against anti-epitope tag antibodies and the target protein. Detailed antibody-staining strategies for each model protein are described in Supporting Information. Fluorescence values representing expression and target binding of the labeled yeast cells were measured using an Accuri C6 flow cytometer (BD Biosciences). Data were collected from 10,000 cells and analyzed using FlowJo software (Treestar Inc.). Full binding titrations were fit as a four-parameter sigmoidal curve using KaleidaGraph (Synergy Software) to calculate equilibrium dissociation constants (KD) from three technical replicates of each fit point.

Sortase bioconjugation reaction. To measure sortase bioconjugation activity and expression on yeast, the plasmids co-expressing sortase A 7M and the LPETGG substrate sequence with various linkers (pCL-Srt-SS, pCL-Srt-LS, and pCL-Srt-cGFP-LS (FIG. 3B)) were transformed into EBY100 yeast cells. Single colonies were grown overnight in SD-CAA at 30° C. Cultures were centrifuged for 2 min at 12,000×g and resuspended to a final OD₆₀₀=10 in fresh SD-CAA. 30 μl of this suspension was used to inoculate 270 μl of the appropriate media containing SD-CAA or SG-CAA with or without 25 mM Azp. Cells were then cultured overnight at 20° C. in 5-ml round-bottom tubes (Corning, 14-959-2). 15 μl of each of these cultures was used for analysis. Samples were washed three times in PBSA and resuspended in PBSA containing 500 μM Sulfo-dibenzocyclooctyne-biotin conjugate followed by incubation for 3 h at room temperature with agitation. These samples were washed three more times with PBSA and resuspended in a 1:500 dilution of both Avidin-PE (Thermo Fisher Scientific, A2660) and chicken anti-c-Myc (Thermo Fisher Scientific A21281) in PBSA for 30 min at room temperature. Samples were washed three more times and used directly for analysis, or resuspended in a 1:250 dilution of AlexaFluor 488 goat anti-chicken IgY (Thermo Fisher Scientific, A11039) and incubated on ice for 20 min. Cells were then washed twice and analyzed. To detect expression and sortase activity, samples were resuspended in 200 μl PBSA and analyzed on a Guava flow cytometer (Millipore). Data were collected from 5,000 cells and analyzed using FlowJo software.

Plasmids construction. The parent pTMY vector was first modified to express a new (Gly₄Ser)₃ linker at the C-terminus of Aga2p as described in the manuscript. The fusion construct expression cassettes were then engineered as follows to streamline binding assays and enzymatic bioconjugation studies, and also to improve usability in future applications.

pCL vectors for binding assays. For binding assays using pCL plasmids, yEGFP was expressed as a fusion to the N- or C-terminus of Aga2p, and used as an indicator for expression levels of the fusion construct on yeast surface. Cloning was used to introduce yEGFP at the N-terminus of Aga2p through NheI and MluI restriction sites (pCL-nGFP; FIG. 5A), leaving the C-terminus of Aga2p available to display a protein of interest. When designing pCL cloning primers containing a MluI site, it is important to include an additional base pair (guanine was used in this study) immediately preceding the MluI site to prevent a reading frame shift. The C-terminal portion of Aga2p was modified to enable display of wild-type Axl Ig1, an engineered Axl variant MYD1 or scFv D1.3. Open reading frames encoding for these proteins were cloned downstream of a second (Gly₄Ser)₃ linker, between AvrII and SpeI restriction sites. A c-Myc tag was included at the C-terminus of the protein of interest to generate pCL-nGFP-Aga2p-Axl (abbreviated pCL-Axl) or pCL-nGFP-Aga2p-D1.3. (abbreviated pCL-D1.3). In addition, a Factor Xa cleavage site and a HA tag were included as handles for protein characterization, if desired. For general use, yEGFP could be replaced by another yeast-optimized fluorescent protein using the NheI and MluI restriction sites, and an alternative protein of interest can be cloned into the vector through the AvrII and SpeI restriction sites.

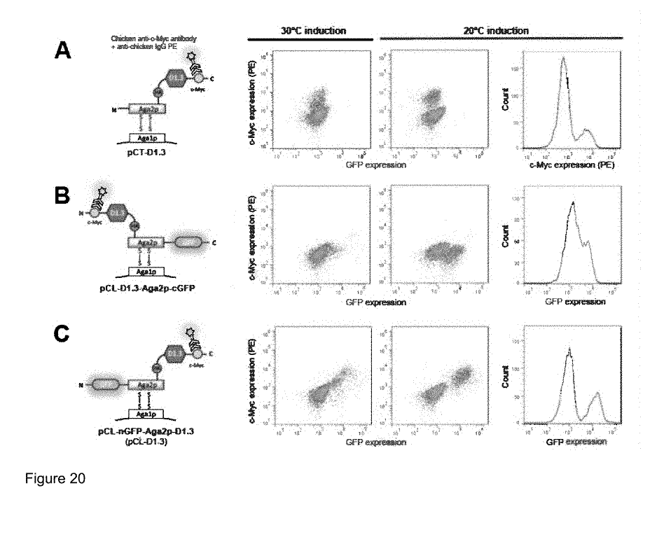

To display a protein of interest at the N-terminus of Aga2p, yEGFP was first cloned at the C-terminus of Aga2p, after the (Gly₄Ser)₃ linker, using XmaI and SpeI restriction sites (pCL-cGFP; FIG. 20B). NK1 was cloned upstream of the first (Gly₄Ser)₃ linker located N-terminal to Aga2p, using NheI and MluI restriction sites to generate pCL-NK1-Aga2p-cGFP (abbreviated pCL-NK1). The vector included a HA tag located upstream and a c-Myc tag located downstream of NK1 to compare protein expression levels measured from epitope-binding antibodies or yEGFP. For general use, yEGFP could be replaced by another yeast-optimized fluorescent protein using the XmaI and SpeI restriction sites, and an alternative protein of interest can be cloned into the vector through the NheI and the MluI restriction sites.

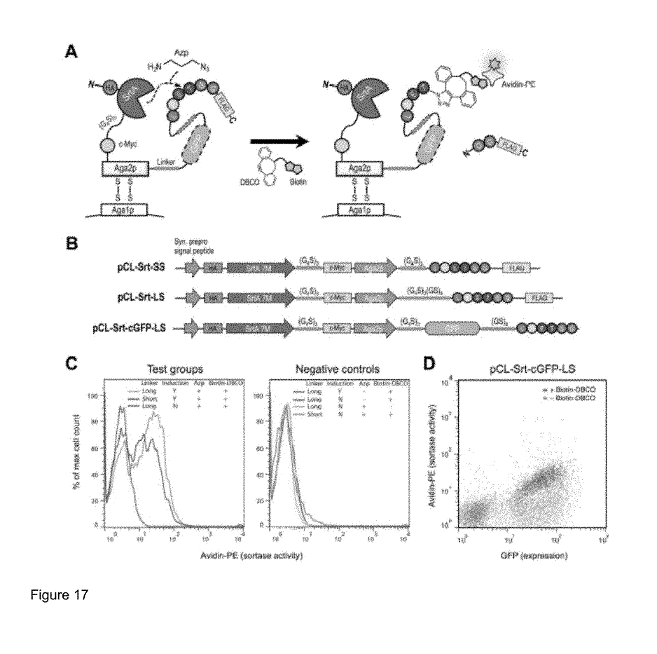

pCL vectors for enzymatic assays. The engineered sortase variant 7M and its substrate sequence LPETGG were cloned into the pCL vector along with different linker strategies: a short linker (15 aa; (Gly₄Ser)₃), a long linker (42 aa; (Gly₄Ser)₃-(Gly₂Ser)₉), or yEGFP plus a long linker ((Gly₄Ser)₃-yEGFP-(Gly₂Ser)₉) (FIG. 17). The open reading frame encoding for sortase 7M was cloned into pCL-cGFP (described above), upstream of Aga2p using NheI and MluI restriction sites. Then, each linker and the LPETGG sequence were cloned downstream of Aga2p between XmaI and SpeI restriction sites. For plasmids with a short or long linker, a FLAG epitope tag was included to validate expression of the linker and LPETGG fusion. Here, nucleotides encoding for the LPETGG and the FLAG tag sequences were codon-optimized for S. cerevisiae as TTGCCAGAAACTGGTGGT and GACTACAAAGACGATGATGACAAG, respectively. The resulting recombinant plasmids are termed pCL-Srt-SS (Short linker plus Substrate sequence), pCL-Srt-LS (Long linker plus Substrate sequence), and pCL-Srt-cGFP-LS (C-terminal GFP plus Long linker and Substrate sequence) (FIG. 17B).

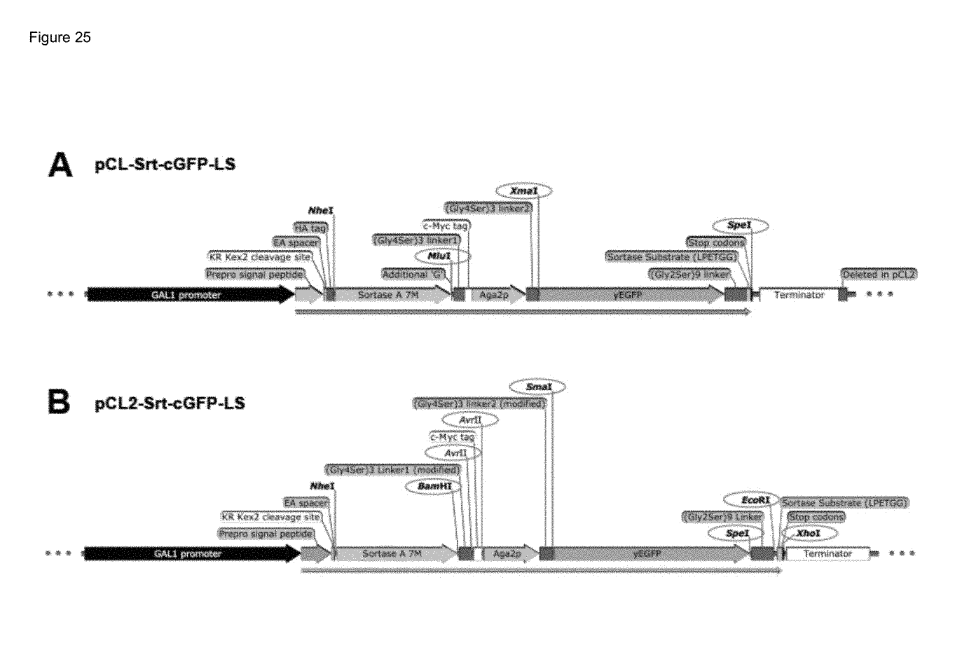

pCL2: An optimized pCL vector for facile and modular cloning. The pCL vector was further optimized (termed pCL2) to add versatility and enhance compatibility with current yeast display platforms. As an example, the vector pCL-Srt-cGFP-LS was rebuilt to generate the pCL2-Srt-cGFP-LS vector (FIG. 25). In the pCL2 construct, the MluI restriction site originally located downstream of the sortase sequence was exchanged with BamHI, a restriction site that is compatible with the widely used pCTCON2 vector. This exchange also eliminates the need for an extra base pair preceding the former MluI site, which was required to keep the translated gene in frame. In addition, two AvrII restriction sites were inserted flanking the c-Myc tag, which allows optional removal of the tag using a single restriction enzyme digestion and re-ligation. Insertion of a SpeI restriction site downstream of the yEGFP and insertion of an EcoRI site upstream of the sortase substrate sequence simplifies exchange of fluorescent proteins or Cterminal elements, respectively. The HA tag was removed from the N-terminus of the entire expression cassette to streamline the construct and to facilitate engineering of the N-terminal domain of a protein-of-interest. Finally, to minimize potential homologous recombination between the (Gly₄Ser)₃ linker regions, these codon sequences were randomized to reduce nucleotide sequence similarity.

Binding assays. scFv D1.3 binding to lysozyme. To evaluate the binding affinity of lysozyme to scFv D1.3 expressed using the pCL or pCT yeast display systems, 1×10⁵ induced yeast cells were incubated with varying concentrations of biotinylated lysozyme (Sigma, L0289) in PBSA for 12 h at room temperature. Yeast expressing scFv D1.3 using the pCT-D1.3 vector were incubated with a 1:250 dilution of chicken anti-c-Myc antibody (Thermo Fisher Scientific, A21281) in PBSA for 30 min at 4° C. Cells were washed with PBSA and incubated with a 1:100 dilution of AlexaFluor 488-labeled goat anti-chicken IgY (Thermo Fisher Scientific, A11039) and a 1:50 dilution of streptavidin-PE (BioLegend, 405204) for 15 min at 4° C. Cells were then washed with PBSA prior to analysis by flow cytometry. Yeast expressing the pCL-nGFP-Aga2p-D1.3 (pCL-D1.3) plasmid were stained with a 1:50 dilution of streptavidin-PE for 15 min at 4° C. and washed with PBSA prior to flow cytometry analysis.

Axl Ig1 binding to Gas6. Similar to the scFv D1.3-lysozyme binding assay, the Gas6 binding affinity of Axl Ig1 expressed using the pCL or pCT vectors were measured by incubating 1×10⁵ induced yeast cells with varying concentrations of His6-tagged Gas6 [8] in PBSA for 17 h at room temperature. For yeast expressing Axl Ig1 using the pCL vector, cells were incubated with a 1:100 dilution of anti-His Tag IgG Hilyte Fluor 555 (Anaspec, AS-61250-H555) for 20 min at 4° C., washed in PBSA, and analyzed by flow cytometry. For yeast transformed with pCT-nGFP-Aga2p-Axl (pCL-Axl), induced cells were incubated with a 1:500 dilution of chicken anti-c-Myc antibody (Thermo Fisher Scientific, A21281) for 30 min at 4° C. Cells were washed with PBSA and secondary antibody labeling was carried out by incubating with a 1:100 dilution of mouse anti-His Tag IgG Hilyte Fluor 555 and a 1:100 dilution of goat anti-chicken IgY AlexaFluor 555 (Thermo Fisher Scientific, A21437) for 20 min at 4° C. Cells were then washed with PBSA and analyzed by flow cytometry.

NK1 binding to Met. To measure the binding affinity of Met receptor to NK1 expressed using the pCL or pTMY vectors, 5×10⁴ induced yeast cells were incubated with various concentrations of recombinant human Met-Fc (R&D Systems, 358-MT-100) for 6 h at 4° C. in PBSA100 (PBSA supplemented with additional 100 mM NaCl). After incubation, yeast cells displaying NK1 using the pCL-NK1-Aga2p-cGFP (pCL-NK1) plasmid were incubated with PBSA100 containing a 1:50 dilution of AlexaFluor 647 labeled goat anti-human IgG (Thermo Fisher Scientific, A21445) for 20 min at 4° C., washed and analyzed by flow cytometry. For yeast transformed with pTMY-NK1, after incubation with Met-Fc, cells were incubated with PBSA100 containing a 1:20 dilution of mouse anti-HA antibody (Cell Signaling Technology, 2367) for 1 h at 4° C. Cells were washed and then incubated in PBSA100 containing a 1:20 dilution of goat anti-mouse IgG PE (Sigma, P9287) for 20 min at 4° C., followed by incubation with PBSA100 containing a 1:50 dilution of AlexaFluor 647 goat anti-human IgG for 20 min at 4° C. Cells were washed and analyzed by flow cytometry.

Antibody and target protein dissociation with washing steps. To compare changes in expression signals due to multiple washing steps performed during binding assays, experiments were designed to mimic various antibody staining strategies: the original condition with a minimal number of staining/washing steps; 1 additional wash for a two-step secondary antibodies staining; and 2 additional washes for a tertiary antibody staining strategy. Here, pCT-Axl and pCL-Axl vectors were used for demonstration.

Yeast cells transformed with pCT-Axl were induced and incubated with Gas6 for 17 h at room temperature. After incubation, cells were washed one time with 1 ml of PBSA and then incubated with 50 μl of PBSA containing a 1:500 dilution of chicken anti-c-Myc antibody (Thermo Fisher Scientific, A21281) for 30 min at 4° C. Following another wash with 1 ml of PBSA, secondary antibody labeling was carried out in 50 μl PBSA containing a 1:100 dilution of mouse anti-His Tag IgG Hilyte Fluor 555 (Anaspec, AS-61250-H555) and AlexaFluor 555 goat antichicken IgY (Thermo Fisher Scientific, A21437) for 20 min at 4° C. Cells were then analyzed by flow cytometry (Accuri C6, BD Biosciences) after the final wash (three washes in total). For the additional washing conditions, one or two incubation/wash steps were added (incubated in 50 μl PBSA and washed with 1 ml PBSA) between the anti-c-Myc primary antibody staining step and the secondary antibodies staining step.

Due to constitutive yEGFP expression, antibody staining conditions are simplified using the pCL vector. Yeast cells containing pCL-Axl were induced, incubated with Gas6 for 17 h at room temperature, washed one time, and then labeled with secondary antibody in 50 μl PBSA containing a 1:100 dilution of mouse anti-His Tag IgG Hilyte Fluor 555. Only two wash steps are necessary in binding assays performed on proteins displayed using the pCL system, including the final wash before flow cytometry analysis. For the additional washing conditions, one or two incubation/wash steps were added between the target-binding step and the secondary antibody staining step.

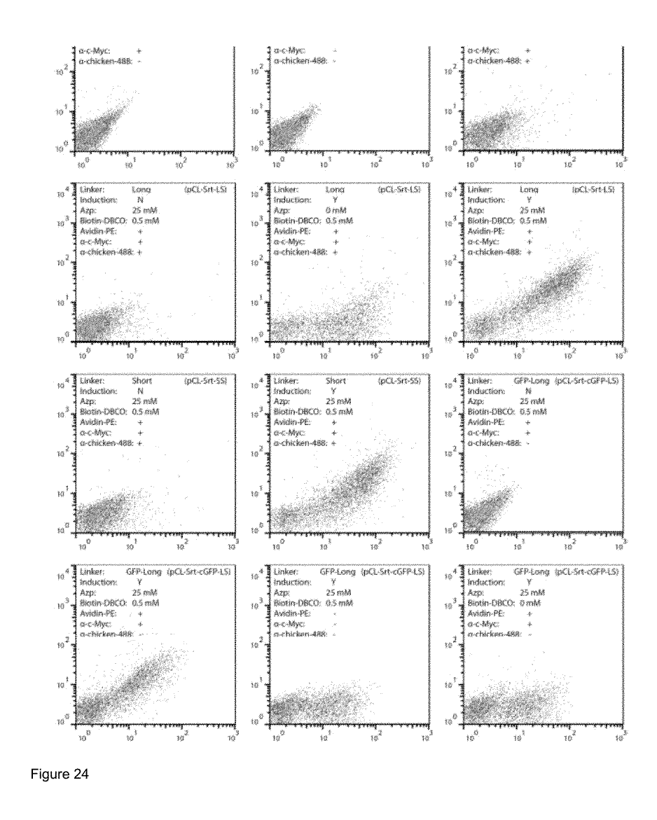

To compare the binding and expression signal changes in the pCT and pCL groups, the mean signal values were normalized to the original condition and shown as bar graphs (FIG. 24B). For statistical analysis, p-values were calculated by Student's t-test (one-tailed, paired).

Stability of yEGFP expression levels. To measure the stability of the yEGFP signal over time, an assay was performed with 5×10⁴ induced yeast cells transformed with pCL-nGFP-Aga2p-Axl. The cells were incubated in 50 μl of PBSA at room temperature and green fluorescence from yEGFP was measured using flow cytometry (Guava, Millipore) at 0, 12, 24, 36, 48, and 72 h in triplicate.

### Results

Dual protein expression vector design. Conventional yeast surface display strategies express a protein of interest as a fusion to the Aga2p subunit at either at the C-terminus (e.g. pCT and pYD1 (Invitrogen) vectors) or N-terminus (e.g. pTMY, pYD5, and pCHA vectors) (FIG. 1A). Our dual protein yeast surface display vector (termed pCL) extends this system, displaying Aga2p flanked by two full-length proteins, one at the N-terminus and one at the C-terminus (FIG. 1B). In the pCL system, the first displayed protein is inserted between a synthetic α-factor prepro signal peptide and the mature Aga2p (without its original signal peptide), fused to the N-terminus of Aga2p (FIG. 15C). The second displayed protein is inserted downstream of Aga2p and fused at the C-terminus of the mature protein. The dual protein expression cassette is led to the yeast secretory pathway by the prepro signal peptide and exported to the cell surface after being processed by the KEX2 endopeptidase that cleaves the C-terminus of Lys-Arg (KR) sequence attached at the end of the signal peptide. The inclusion of an EA (Glu-Ala) peptide spacer helps with efficient KEX2 cleavage of the fusion protein. For the proof-of-concept studies described here, epitope tags were included on both the N- and C-terminal sides of Aga2p, as in the traditional pCT-based protein expression system, to validate expression of the fusion construct on the yeast cell surface.

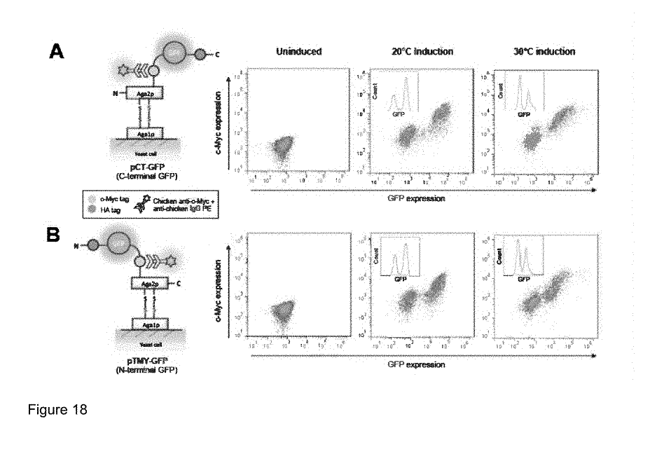

A fluorescent protein fusion enables measurement of protein expression levels on the yeast cell surface. The original yeast display system uses antibody-labeling of N- or C-terminal epitope tags to quantify yeast surface expression levels by flow cytometry. In the pCL system, a fluorescent protein is fused to the N- or C-terminus of the Aga2p protein as a handle to measure expression of the entire fusion construct on yeast surface. The fluorescent protein utilized in our construct is a yeast-codon-optimized enhanced GFP (yEGFP), although the modularity of the system theoretically allows for the use of any yeast-optimized fluorescent protein to suit the characteristics (brightness, wavelength) desired by the user. A previous study showed that yEGFP is well-expressed as a C-terminal fusion to Aga2p on the widely used pCT vector. To allow for a more flexible design, we confirmed that yEGFP is also functionally expressed at the N-terminus of Aga2p when it is inserted between the signal peptide and the mature protein (FIG. 18).

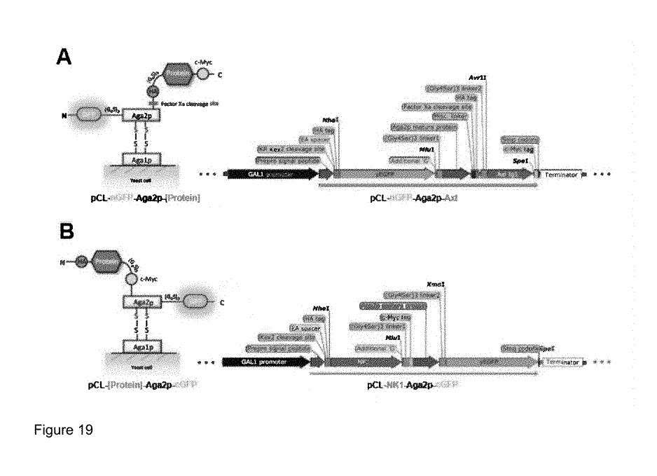

Two versions of the dual protein expression vector were constructed: pCL-nGFP (yEGFP fused to the N-terminus of Aga2p) and pCL-cGFP (yEGFP fused to the C-terminus of Aga2p) (FIG. 19). When analyzed using two-parameter flow cytometry scatter plots, both the N-terminal and the C-terminal yEGFP fusions showed distinct separation between expression-positive and expression-negative yeast cell populations (FIG. 18). For both pCL-nGFP and pCL-cGFP, induction of fusion protein expression at 20° C. showed higher levels of GFP-positive cells compared to the 30° C. induction conditions. Therefore, all of the analyses this study were performed with yeast cells induced at 20° C.

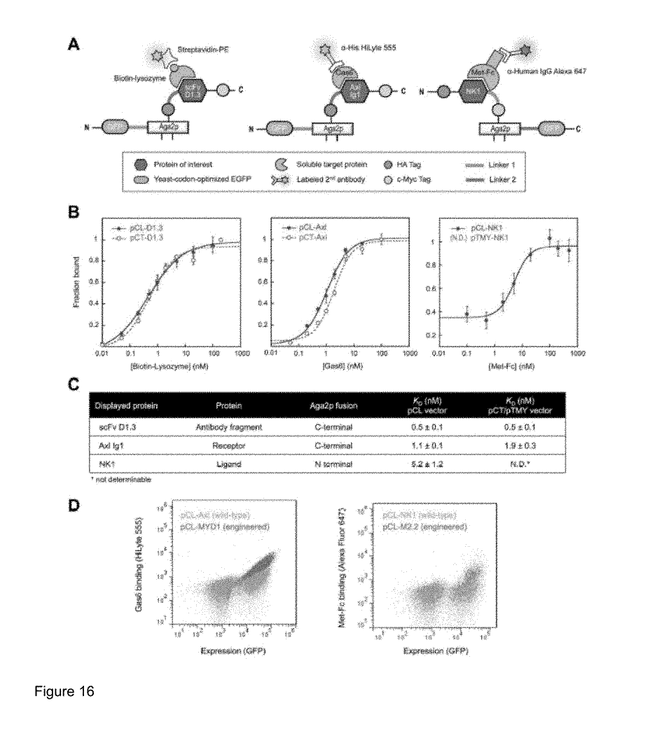

Dual protein expression enables quantification of binding interactions on the yeast cell surface. We evaluated the reliability of the pCL-based strategy for measuring protein-protein binding interactions by testing three model proteins: 1) scFv D1.3, a murine antibody fragment that binds hen egg lysozyme, 2) Axl Ig1, a wild-type receptor domain that binds its cognate ligand growth arrest specific 6 (Gas6), and 3) NK1, a natural fragment of the N-terminal and first kringle domain of human hepatocyte growth factor ligand that binds Met receptor. The scFv D1.3 and Axl Ig1 proteins were tethered through their N-terminus to the C-terminus of Aga2p, which in turn has yEGFP as an N-terminal fusion (FIG. 16A, left and middle panels, pCL-nGFP-Aga2p-D1.3 (abbreviated pCL-D1.3) and pCL-nGFP-Aga2p-Axl (abbreviated pCL-Axl); FIGS. 19A and 17). To showcase the breadth of the system, NK1 was tethered through its C-terminus to the N-terminus of Aga2p, which has a C-terminal yEGFP fusion (FIG. 16A, right panel, pCL-NK1-Aga2p-cGFP (abbreviated pCL-NK1); FIG. 19B). A liberated N-terminus of NK1 has been shown to be a preferable orientation for this protein.

Binding assays carried out with the dual protein display system are streamlined due to the constitutive fluorescence of yEGFP as a yeast surface expression read-out. After incubation with a soluble target protein, the induced yeast cells only require a single staining step with fluorophore-labeled secondary antibody against the binding target. This can be further reduced to a single-step binding assay when a soluble target is covalently labeled with a fluorescent dye. In contrast, assays involving conventional yeast display vectors require additional primary (and often secondary or sometimes tertiary), antibody staining steps for measuring protein expression levels through an N- or C-terminal epitope tag, resulting in multiple incubation and cell washing steps.

Using the simplified workflow described above, pCL-based yEGFP expression vectors were used to measure binding affinities of the three model proteins to their respective targets. The constructs pCL-D1.3, pCL-Axl and pCL-NK1 were expressed on the yeast surface and incubated with a corresponding binding partner in solution: biotinylated lysozyme (for scFv D1.3), Gas6 (for Axl Ig1), or Met-Fc (for NK1). Secondary antibodies labeled with fluorescent dyes were used to measure target-binding signals by flow cytometry (FIG. 16A). All the induced yeast cells showed a distinct GFP-positive population in scatter plots, enabling clear gating of the expressing population from which to quantify binding. Representative flow cytometry scatter plots for pCL-Axl at various Gas6 target concentrations are shown in FIG. 18.

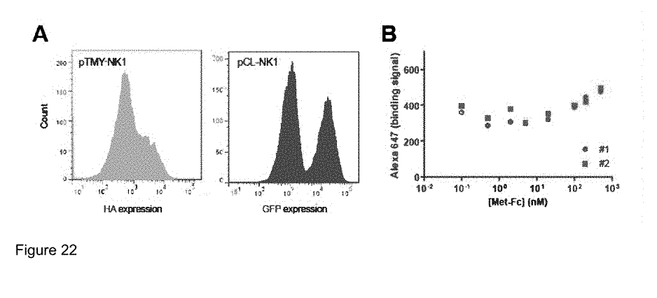

The results generated with the pCL system were compared with the results obtained from the epitope-tag-expressing conventional yeast display vectors, pCT (c-Myc expression) or pTMY (HA expression). Binding assays carried out with pCL-D1.3 and pCL-Axl recapitulated equilibrium binding curves and respective affinities obtained with pCT-D1.3 and pCT-Axl (FIG. 16B, left and middle panels, and C). Measurement of pTMY-based yeast display of NK1 showed weak cell surface expression and negligible binding to Met-Fc (FIG. 22), in agreement with our previous study. In contrast, pCL-NK1 enabled an equilibrium dissociation constant against Met-Fc to be measured (FIG. 16, right panel). As observed in the NK1 scatter plots, pCL-NK1 generates a notably higher expression signal compared to antibody epitope tag staining of pTMY-NK1, permitting measurement of Met-Fc binding signals in a dose-dependent manner (FIG. 22).

A fusion construct displayed using the pCL vector (pCL-Axl) showed a strong linear correlation (R²=0.75) between the yEGFP signal and the c-myc epitope tag expression signal on the same yeast cell (FIG. 24A). As a final example, we show that the pCL system can differentiate wild-type proteins and engineered high affinity variants expressed on the yeast cell surface as measured by flow cytometry scatter plots (FIG. 16D). These results demonstrate the applicability of the vectors for library construction and screening required for combinatorial protein engineering.

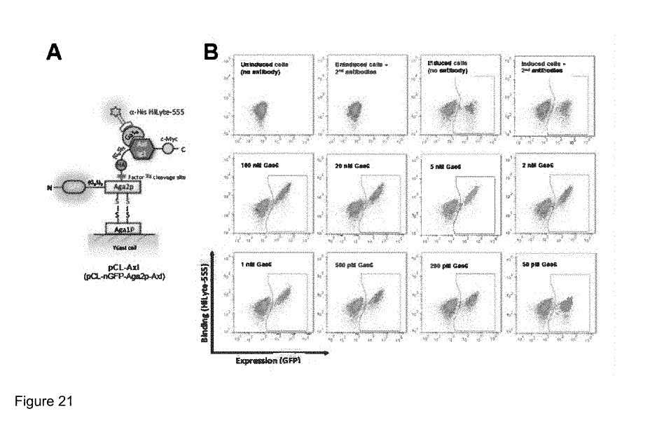

Additional considerations of the pCL display system. In addition to the time and cost benefit of not having to use anti-epitope antibodies to measure expression levels, the pCL-based yeast display strategy avoids a decrease in fluorescence signals which is derived from antibody or target dissociation due to multiple wash steps used in the traditional pCT system. As shown in FIG. 21B, each additional cell washing step significantly decreased c-Myc expression signals in the pCT group, demonstrating dissociation of the anti-c-Myc primary antibody from the cell yeast surface. In contrast, GFP expression signals remained constant in the pCL group, regardless of additional washing steps. Eliminating one or two washing steps for sample preparation may be especially beneficial for studying a protein with low expression or weak target binding on the yeast cell surface. Moreover, the fluorescent signal from yEGFP expression was stable at room temperature when analyzed over a 72 h period (FIG. 9C), which spans the time window generally used for measuring kinetic dissociation rates or screening a yeast-displayed library based on kinetic off-rate parameters.

Dual protein expression enables quantification of enzyme-substrate bioconjugation reactions on the yeast cell surface. We next investigated whether the pCL system could be applied to quantify the reaction between a bioconjugation enzyme and its substrates. In this case we developed a pCL-based enzyme-substrate system to measure sortase activity on the yeast cell surface, a simplified design from the elegant display technology previously developed by the Liu lab. Sortase is a transpeptidase enzyme that has widespread use in bioconjugation reactions. The sortase enzyme from Staphylococcus aureus and its engineered derivatives 5M and 7M recognize a LPXTG sequence and exchange the terminal glycine for a variety of nucleophiles, such as glycine-terminal peptides, hydrazides, and various primary amines. We previously showed that by using non-natural amine nucleophiles, we could label proteins in living E. coli cells.

We introduced the calcium-independent version of sortase, 7M, into the pCL vector and tested its ability to incorporate the non-biological amine 3-azido-1-propanamine (Azp) into LPETGG sequences on the surface of yeast. Sortase 7M was fused to N-terminus of Aga2p, followed by: 1) a short linker, 2) a long linker, or 3) yEGFP plus a long linker C-terminal to Aga2p (FIGS. 3A and B). Each of these constructs contained a C-terminal LPETGG substrate sequence so that the sortase variant could carry out its enzymatic modification. The linkers were intended to test accessibility of sortase to LPETGG with different flexibilities and distances between the enzyme and the substrate.

The constructs were transformed into yeast and induced at 20° C. in the presence of Azp in the induction media. In each case, the sortase 7M variant and the linker-LPETGG were successfully expressed on yeast as shown by c-Myc staining or yEGFP fluorescence (FIG. 25). Moreover, each enzyme-substrate construct was able to facilitate conjugation with Azp, followed by a Copper-free Click reaction between the incorporated Azp and sulfo-biotin DBCO. Biotin-conjugated 7M fusions were detected by staining with PE-labeled avidin (FIG. 17C, left). Negative controls lacking Azp or reactions carried out with yeast cultured in non-inducing media suggested that the Click reaction was specific to Azp and the LPETGG substrate sequence (FIG. 17C, right). Both the long linker variant (pCL-Srt-LS) and the short linker variant (pCL-Srt-SS) showed substantial bioconjugation, indicating that they allow sufficient proximity of the C-terminal substrate to the N-terminal enzyme (FIG. 17C, left).

Finally, a bioconjugation reaction specific to sulfo-biotin DBCO was also observed when yEGFP was included in the construct (pCL-Srt-cGFP-LS; FIGS. 17B and D). 7M-expressing cells were readily distinguished by yEGFP fluorescence, and active sortase was again detected by avidin-PE using flow cytometry. Based on the close proximity of N-terminus and C-terminus of GFP, this construct does not interfere with LPETGG substrate access to the sortase active site. Similar to the binding assays described above, the GFP fusion construct has the advantage of not requiring a second antibody stain for expression, facilitating rapid analysis of cell surface chemistry.

In this study, we showed that the pCL yeast display system enables co-expression of two proteins: a protein of interest and a fluorescent protein, or an enzyme and substrate pair, to facilitate protein binding assays or catalytic reactions, respectively. Considering the relatively high price of antibodies against epitope tags (such as c-Myc, FLAG, and HA), the pCL vectors containing yEGFP provide a cost-effective and simple means for measuring protein expression on the yeast cell surface. Moreover, we showed that the yEGFP signals generated from the pCL system are stable and correlate with expression levels of co-expressed proteins of interest, thus providing an alternative analysis method for yeast surface display. While direct fusion of a yEGFP to the N- or C-terminus of the displayed protein in FIG. 1A is possible, the pCL system uses Aga2p as a spacer to minimize the effects of a bulky fluorescent protein on sterics and folding. Although the current vectors used in this study utilize yEGFP to quantify a protein expression, which shows optimal induction of yeast expression at 20° C., other fluorescent proteins with improved expression and stability could be used in future studies. We have also created a modified pCL vector (termed pCL2) containing codon-optimized linkers and additional restriction sites, suitable for facile and modular cloning and library construction (FIG. 25).

The pCL dual protein display system also enables measurement of enzyme-mediated bioconjugation on the surface of yeast. The co-expression of sortase and the substrate sequence as a single fusion protein imparts a distinct advantage for incorporation of non-natural nucleophiles in yeast over a previously developed yeast display system for bioconjugation. First, the pCL-based approach does not require solid-phase peptide synthesis of substrate sequences, and has no need for a secondary enzyme (in the previous study, phosphopantotheinyl transferase) to attach sortase substrates to the surface. Second, the pCL vector can be used with widely available EBY100 yeast, rather than the modified strain required in the previous system. Third, the platform is flexible, and a library of enzyme variants or peptide substrates could be created and screened using the pCL system. Thus, yeast display using the pCL dual protein display system provides simplicity and generalizability in the interrogation of enzymatic activity as well as protein-protein binding interactions. In addition, the pCL system has potential applications to studies where display of multiple proteins on the same yeast cell is desired, including split fluorescent protein engineering or sequential assembly of multi-enzyme cascades.

Sequences

| | |
|---|---|
| SrtA7M | SEQ ID NO: 25 |
| WT SrtA Δ59 | SEQ ID NO: 26 |
| pRhaGG | SEQ ID NO: 27 |
| MBP-srt | SEQ ID NO: 28 |
| Fn10-His-srt | SEQ ID NO: 29 |
| 5f7-His-srt | SEQ ID NO: 30 |
| Trx-5f7-His-srt | SEQ ID NO: 31 |
| sfGFP-His-srt | SEQ ID NO: 32 |
| His-sfGFP-srt | SEQ ID NO: 33 |
| GST-His-srt | SEQ ID NO: 34 |

What is claimed is:

1. A method of conjugating an amine substrate on the C-terminus of a target protein, the method comprising the steps of:
(a) providing the target protein with a C-terminal sortase recognition sequence;
(b) contacting the target protein with an amine substrate; in the presence of an engineered sortase enzyme under conditions suitable for the sortase to conjugate the substrate to the target protein.

2. The method of claim 1, wherein the amine substrate is other than an amino acid.

3. The method of claim 1, wherein the sortase enzyme is SrtA7M.

4. The method of claim 1, wherein the substrate is selected from: 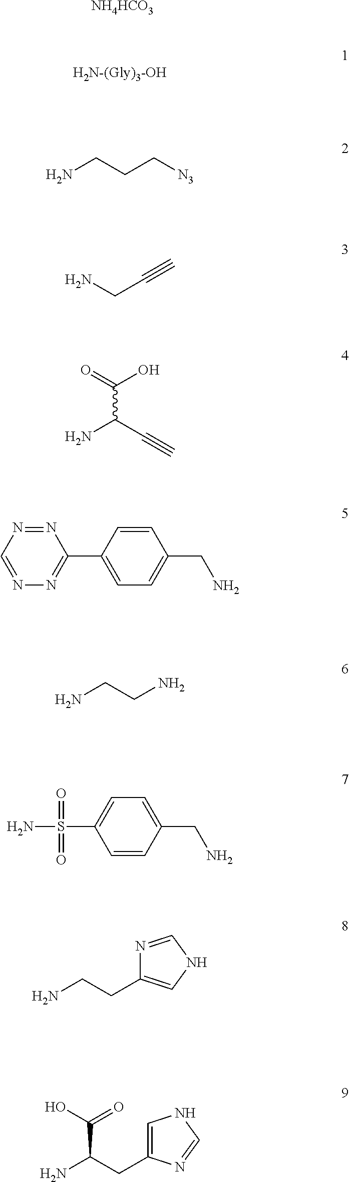
or an analog or derivative thereof.

5. The method of claim 1, wherein the contacting is performed in vitro.

6. The method of claim 1, wherein the contacting is performed in vivo.

7. The method of claim 6, wherein the in vivo contacting is in a microbial cell.

8. The method of claim 1, wherein the sortase recognition sequence is LPETG.

9. A kit for use in the method of claim 1.


SEQUENCE LISTING (34 sequences)

SEQ ID NO: 1 - DNA, 40 bp, Artificial Sequence
  Synthetic nucleotide
accattggtctctgccgttacttggtgatacgagtctgcg

SEQ ID NO: 2 - DNA, 45 bp, Artificial Sequence
  Synthetic nucleotide
accattggtctcaggtaaaaatgaaaatcgaagaaggtaaactgg

SEQ ID NO: 3 - DNA, 52 bp, Artificial Sequence
  Synthetic nucleotide
gaggtgtaagctttgcaaaagaaaatcaatcactagatgatcaaaatatttc

SEQ ID NO: 4 - DNA, 52 bp, Artificial Sequence
  Synthetic nucleotide
gaaatattttgatcatctagtgattgattttcttttgcaaagcttacacctc

SEQ ID NO: 5 - DNA, 64 bp, Artificial Sequence
  Synthetic nucleotide
accattggtctctgccgttagccaccggtttccggcaggctgcccttggtgatacgagtctgcg

SEQ ID NO: 6 - DNA, 36 bp, Artificial Sequence
  Synthetic nucleotide
accattggtctctcatgcaagctaaacctcaaattc

SEQ ID NO: 7 - DNA, 31 bp, Artificial Sequence
  Synthetic nucleotide
tagcaaggatccgttagcagccggatctcag

SEQ ID NO: 8 - DNA, 59 bp, Artificial Sequence
  Synthetic nucleotide
accattggtctctggtaaaaatgcatcaccatcaccatcaccgtaaaggcgaagagctg

SEQ ID NO: 9 - DNA, 64 bp, Artificial Sequence
  Synthetic nucleotide
accattggtctctgccgttaaccggtttccggcaggctgcctttgtacagttcatccataccat

SEQ ID NO: 10 - DNA, 41 bp, Artificial Sequence
  Synthetic nucleotide
accattggtctctggtaaaaatgcgtaaaggcgaagagctg

SEQ ID NO: 11 - DNA, 76 bp, Artificial Sequence
  Synthetic nucleotide
acaggtctctgccgttaaccaccggtttccggcaggtgatggtgatggtgatgtttgtacagttcatccataccat

SEQ ID NO: 12 - DNA, 39 bp, Artificial Sequence
  Synthetic nucleotide
attggtctctggtaaaaatgcaagctaaacctcaaattc

SEQ ID NO: 13 - DNA, 32 bp, Artificial Sequence
  Synthetic nucleotide
acaggtctctgccgttagcagccggatctcag

SEQ ID NO: 14 - DNA, 32 bp, Artificial Sequence
  Synthetic nucleotide
tcaaggtctcgcggctgctgctggtctgctgc

SEQ ID NO: 15 - DNA, 41 bp, Artificial Sequence
  Synthetic nucleotide
gtaaggtctcttacctctccttcttaaagttaaacaaaatt

SEQ ID NO: 16 - DNA, 43 bp, Artificial Sequence
  Synthetic nucleotide
accattggtctctggtaaaaatggaagtgcagctggtggaaag

SEQ ID NO: 17 - DNA, 71 bp, Artificial Sequence
  Synthetic nucleotide
acaggtctctgccgttaaccaccggtttccggcaggtgatggtgatggtgatggctgctcacggtcacctg

SEQ ID NO: 18 - DNA, 40 bp, Artificial Sequence
  Synthetic nucleotide
accattggtctctggtaaaaatgctggttccgcgtggatc

SEQ ID NO: 19 - DNA, 74 bp, Artificial Sequence
  Synthetic nucleotide
acaggtctctgccgttaaccaccggtttccggcaggtgatggtgatggtgatgaccgcctttaccttttttacc

SEQ ID NO: 20 - DNA, 44 bp, Artificial Sequence
  Synthetic nucleotide
accattggtctctggtaaaaatgccaccatacaccgttgtttac

SEQ ID NO: 21 - DNA, 69 bp, Artificial Sequence
  Synthetic nucleotide
acaggtctctgccgttaaccaccggtttccggcaggtgatggtgatggtgatgttgcttcccgtttccg

SEQ ID NO: 22 - DNA, 42 bp, Artificial Sequence
  Synthetic nucleotide
accattggtctcaggtaaaaatgagcgataaaattattcacc

SEQ ID NO: 23 - DNA, 34 bp, Artificial Sequence
  Synthetic nucleotide
atatcaggtctctcagacttgtcgtcgtcgtcgg

SEQ ID NO: 24 - DNA, 42 bp, Artificial Sequence
  Synthetic nucleotide
accattggtctcttctggcatggaagtgcagctggtggaaag

SEQ ID NO: 25 - DNA, 471 bp, Homo sapiens
atgcaagctaaacctcaaattccgaaagataaatcaaaagtggcaggctatattgaaattccagatgctgatattaaagaaccagtatatccaggaccagcaacacgcgaacaattaaatagaggtgtaagctttgcaaaagaaaatcaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagaaacgttaagccaacagctgtagaagttctagatgaacaaaaaggtaaagataaacaattaacattaattacttgtgatgattacaatgaagagacaggcgtttgggaaacacgtaaaatctttgtagctacagaagtcaaactcgagcaccaccaccaccaccactga

SEQ ID NO: 26 - DNA, 471 bp, Homo sapiens
atgcaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccactga

SEQ ID NO: 27 - DNA, 3054 bp, Homo sapiens
atcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaagaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtatgaaattcttttaaggaggtaagagacctcccctatcagtgatagagattgacatccctatcagtgatagagatactgagcacggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggatctgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaggtctcgcggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcttcttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgcagaaaggcccacccgaaggtgagccaggtgattacatttgggccctcattagaaaaactcatcgagcatcaaatgaaattgcaatttattcatatcaggatt

SEQ ID NO: 28 - DNA, 1140 bp, Homo sapiens
atgaaaatcgaagaaggtaaactggtaatctggattaacggcgataaaggctataacggcctggctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagggcagcctgccggaaaccggtggctaa

SEQ ID NO: 29 - DNA, 360 bp, Homo sapiens
atgctggttccgcgtggatcctctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactccgcgtggagactggaacgaaggtagcaagccaatttccattaattaccgaacaggtaaaaaaggtaaaggcggtcatcaccatcaccatcacctgccggaaaccggtggttaa

SEQ ID NO: 30 - DNA, 360 bp, Homo sapiens; identical to SEQ ID NO: 29

SEQ ID NO: 31 - DNA, 876 bp, Homo sapiens
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaagtctggcatggaagtgcagctggtggaaagcggcggcggcctggtgcaggcgggcggcagcctgcgcctgagctgcgcggcgagcggcattacctttagcattaacacgatgggctggtatcgccaggcgccgggcaaacagcgcgaactggtggcgctgattatcagcattggcgatacctattatgcggatagcgtgaaaggccgctttaccattagccgcgataacgcgaaaaacaccgtgtatctgcagatgaacagcctgaaaccggaagataccgcggtgtattattgcaaacgctttcgcaccgcggcgcagggcaccgattattggggccagggcacccaggtgaccgtgagcagccatcaccatcaccatcacctgccggaaaccggtggttaa

SEQ ID NO: 32 - DNA, 753 bp, Homo sapiens
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggatctgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaacatcaccatcaccatcacctgccggaaaccggtggttaa

SEQ ID NO: 33 - DNA, 750 bp, Homo sapiens
atgcatcaccatcaccatcaccgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggatctgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaactgccggaaaccggttaa

SEQ ID NO: 34 - DNA, 669 bp, Homo sapiens
atgccaccatacaccgttgtttactttcctgttcgtggtagatgtgccgctcttcgtatgttattggccgatcaaggacaatcatggaaggaagaagttgttactgttgaaacctggcaagaaggttcattaaaggcctcatgtttatacggacaattgccaaagtttcaagatggagatttgaccttataccaatctaacactattcttcgtcatttgggtcgtaccttgggattgtacggaaaggatcaacaagaagctgccttagttgatatggttaacgatggtgttgaggatcttagatgcaaatacatttctcttatatacaccaactacgaagccggaaaggatgattacgttaaggctcttccaggacaattaaagccatttgaaacccttttgtcacaaaaccaaggtggcaaaacctttattgttggagatcaaatttcatttgccgattacaacttgcttgatcttcttcttattcatgaagttttggccccaggatgtcttgatgcttttccattattgtccgcctacgttggtagactttcagctagaccaaaattgaaagccttcttagcctctccagaatacgttaaccttcctattaacggaaacgggaagcaacatcaccatcaccatcacctgccggaaaccggtggttaa